(19) United States
(12) Patent Application Publication
(10) Pub. No.: US 2023/0293735 A1
(43) Pub. Date: Sep. 21, 2023

(54) OXINE-CONTAINING CELL RADIOLABELLING AGENTS

(71) Applicant: KING'S COLLEGE LONDON, London (GB)

(72) Inventors: Francis MAN, London (GB); Philip J. BLOWER, London (GB); Rafael TORRES MARTIN DE ROSALES, London (GB)

(21) Appl. No.: 18/002,761

(22) PCT Filed: Jun. 21, 2021
(86) PCT No.: PCT/EP2021/066865

§ 371 Date: Dec. 21, 2022

(30) Foreign Application Priority Data
Jun. 22, 2020 (GB) 2009512.1

(51) Int. Cl. A61K 51/04; A61K 47/26; A61K 47/12; A61K 47/20; A61K 47/10

(52) U.S. Cl. CPC A61K 51/0482; A61K 47/10; A61K 47/12; A61K 47/20; A61K 47/26

(57) ABSTRACT

The present invention relates to cell radiolabelling agents. The invention provides a method of preparing oxine-containing cell radiolabelling agents, a kit for the preparation of oxine-containing cell radiolabelling agents and a formulation for the preparation of oxine-containing cell radiolabelling agents, in particular ⁸⁹Zr-oxine cell radiolabelling agents.

### FIELD OF THE INVENTION

The present invention relates generally to cell radiolabelling agents. More specifically, the present invention relates to a method of preparing oxine-containing cell radiolabelling agents, a kit for the preparation of oxine-containing cell radiolabelling agents and a formulation for the preparation of oxine-containing cell radiolabelling agents. Particularly, the present invention relates to the preparation of ⁸⁹Zr-oxine, ⁶⁸Ga-oxine, ⁶⁴Cu-oxine, ¹¹¹In-oxine, ⁶⁷Ga-oxine or ⁵²Mn-oxine cell radiolabelling agents. More particularly, the present invention relates to the preparation of ⁸⁹Zr-oxine cell radiolabelling agents.

### BACKGROUND

Cells can be exploited to identify and treat disease. Radiolabelled leukocytes are routinely used to detect sites of infection [1]. For example, stem cells can be delivered to diseased tissue to promote tissue regeneration, and cancer-cell targeted T cells have resulted in high cancer remission rates. [2-4] However, despite remarkable results, very few cell products have been approved for clinical use. One of the main reasons for this lack of progress is a failure to provide sufficient safety and efficacy assurances, which can be linked to a lack of understanding of their in vivo behaviour. Currently, clinical trials are performed without knowledge about the location and fate of cells, making it impossible to adequately monitor and assess safety. For example, in a recent clinical trial, patient deaths due to the immunotherapy exerting effects at the wrong body location [5] could have been prevented if the cells had been traceable and controllable.

An advanced clinical imaging method for tracking cells in vivo at the whole body level is positron emission tomography (PET).[6] In comparison to gamma scintigraphy and SPECT imaging, which are the clinically established cell tracking techniques, PET has superior sensitivity, quantifiability and higher spatial resolution, allowing for the detection of smaller amounts of radioactivity over smaller volumes. Thus, using PET technology would provide a highly sensitive and accurate method to track and spatially quantify therapeutic cells.

Several different positron-emitting radiolabels for cells have been investigated. [¹⁸F]Fluorodeoxyglucose (FDG) is internalized by cells via glucose transporters, but efflux is high and labelling efficiencies are variable; moreover, the short half-life (110 min) of ¹⁸F allows only brief tracking [7-9]. ⁶⁸Ga has been used to label cells [10] although its half-life (68 min) is short. ⁶⁴Cu has a half-life (12.7 h) more suitable for cell tracking and has been efficiently incorporated into cells using lipophilic tracers [11-15], although there is rapid efflux of ⁶⁴Cu from labelled cells.

Zirconium-89 (⁸⁹Zr) is a long half-life positron emitter (t_{½} = 78.4 h, (3+: 23%). In recent years, ⁸⁹Zr-oxine has emerged as a useful radiolabel for the tracking of cells using PET. It has been used to label various cell types including tumour cell lines [16-18], bone marrow and dendritic cells [19-21] and therapeutic T cells [22-24], as well as liposomes [25,26] and extracellular vesicles [32].

However, many potentially useful radiolabels, including ⁸⁹Zr-oxine, ⁶⁸Ga-oxine, ⁶⁴Cu-oxine, ⁶⁷Ga-oxine and ⁵²Mn-oxine are yet to be available commercially or used in a clinical setting. One of the main barriers to the use of agents such as ⁸⁹Zr-oxine, ⁶⁸Ga-oxine, ⁶⁴Cu-oxine, ⁶⁷Ga-oxine or ⁵²Mn-oxine for the radiolabelling of cells in the clinic is the absence of simple, easy to use preparations. Earlier methods for formation of ⁸⁹Zr-oxine, for example, involved bi-phasic chloroform/water extraction. Chloroform was used as a solvent which was then subsequently evaporated and redissolution in DMSO or ethanol [17]. In practice, this reduces the prospects of clinical adoption because it involves many steps, requires meticulous precision during the neutralisation step and involves organic solvents and heating, with an overall radiochemical yield of 60-80%. The method described by Sato et al. starts from ⁸⁹Zr-chloride [19], whereas commercial ⁸⁹Zr is typically produced and shipped as the less-reactive ⁸⁹Zr-oxalate [27,28], requiring an additional conversion step [29]. Leaving this conversion to the final user increases the number of steps, time and operator exposure, and reduces activity concentration. All of these factors greatly restrict the clinical translation and commercial appeal for the routine use of labels such as ⁸⁹Zr-oxine, ⁶⁸Ga-oxine, ⁶⁴Cu-oxine, ⁶⁷Ga-oxine and ⁵²Mn-oxine in clinical trials.

WO 2015/153772 relates to the preparation of a ⁸⁹Zr-oxine complex for labelling cells. However, WO 2015/153772 does disclose a ‘one-step’ method for preparing oxine-containing cell radiolabelling agents. In WO 2015/153772 after a first step of mixing ⁸⁹ZrCl₄ with an oxine solution, a subsequent step of adding alkali to neutralise the mixture is performed. This reduces the prospects of clinical adoption of the formulation because meticulous precision is required during the subsequent neutralisation step. In WO 2015/153772 the radioactive metal is also provided to the oxine solution in a chloride solution. This is less preferable as commercial ⁸⁹Zr is typically produced and shipped in an oxalic acid solution. An addition step of converting commercially available ⁸⁹Zr-oxalate to ⁸⁹ZrCl₄ is required in WO 2015/153772 before the ⁸⁹ZrCl₄ is mixed with an oxine solution.

EP 0017355 is an application relating to a preparation of Indium-111 oxine. However, there is no disclosure of a step where a solution containing a suitable radioactive metal is added to a formulation comprising oxine; a surfactant; a base; and a buffering agent. There is no disclosure of use of a base in the preparation of EP 0017355. The radioactive metal is also added to the preparation in a less preferable chloride solution.

Therefore, there exists a need for the development of a simple method for preparing oxine-containing cell radiolabelling agents which will allow their easy preparation and use in a clinical setting.

The present invention has been devised in light of the above considerations.

### SUMMARY OF THE INVENTION

The present inventors have devised a novel and inventive approach to this problem, which focuses on the formation of oxine-containing cell radiolabelling agents in a single step. The oxine-containing cell radiolabelling agent comprises a radioactive metal complexed with at least one oxine group. In some embodiments, the invention focuses on forming ⁸⁹Zr-oxine, ⁶⁸Ga-oxine, ⁶⁴Cu-oxine, ¹¹¹In-oxine, ⁶⁷Ga-oxine or ⁵²Mn-oxine in a single step. In preferred embodiments, the invention is directed to the formation of ⁸⁹Zr-oxine in a single step. This is in contrast to other approaches which can only be conducted in multiple steps. The invention allows the simple and radiopharmacy-compatible radiolabelling of cells with oxine-containing cell radiolabelling agents, for example for imaging techniques such as SPECT scanning or PET scanning. Advantageously, an oxine-containing cell radiolabelling agent (for example, ⁸⁹Zr-oxine) prepared as described herein is in a stable composition, which can be used immediately or can be stored, for example for up to one week.

The present invention accordingly provides a method, i.e. a ‘one-step’ method, for preparing oxine-containing cell radiolabelling agents. The method preferably comprises adding a solution containing a suitable radioactive metal to a formulation comprising: oxine; a surfactant; a base; and a buffering agent. In some embodiments, the oxine-containing cell radiolabelling agent is ⁸⁹Zr-oxine, ⁶⁸Ga-oxine, ⁶⁴Cu-oxine, ¹¹¹In-oxine, ⁶⁷Ga-oxine or ⁵²Mn-oxine. In some embodiments, the oxine-containing cell radiolabelling agent is ⁸⁹Zr-oxine, ⁶⁸Ga-oxine or ⁶⁴Cu-oxine. In preferred embodiments, the oxine-containing cell radiolabelling agent is ⁸⁹Zr-oxine.

In another aspect of the invention, provided herein is a kit for the preparation of an oxine-containing cell radiolabelling agent. The kit preferably comprises a) a formulation comprising: oxine; a surfactant; a base; and a buffering agent; and b) instructions for use. In some embodiments, the kit is for the preparation of ⁸⁹Zr-oxine, ⁶⁸Ga-oxine, ⁶⁴Cu-oxine, ¹¹¹In-oxine, ⁶⁷Ga-oxine or ⁵²Mn-oxine. In some embodiments, the kit is for the preparation of ⁸⁹Zr-oxine, ⁶⁸Ga-oxine, or ⁶⁴Cu-oxine. In preferred embodiments, the kit is for the preparation of ⁸⁹Zr-oxine. Preferably, the instructions are for the preparation of an oxine-containing cell radiolabelling agent, for example ⁸⁹Zr-oxine, ⁶⁸Ga-oxine, ⁶⁴Cu-oxine, ¹¹¹In-oxine, in accordance with the methods describe herein.

In a further aspect of the invention, provided herein is a formulation for use in the preparation of an oxine-containing cell radiolabelling agent, for example for the preparation of ⁸⁹Zr-oxine, ⁶⁸Ga-oxine, ⁶⁴Cu-oxine, ¹¹¹In-oxine, ⁶⁷Ga-oxine or ⁵²Mn-oxine, said formulation comprising: oxine; a surfactant; a base; and a buffering agent. The formulation of the invention is suitable for use in the methods of preparing oxine-containing cell radiolabelling agents described herein. In some embodiments, the formulation may also comprise a solvent, preferably water. In some embodiments the formulation may be freeze-dried, to be reconstituted with a solvent before use.

In a further aspect of the invention, provided herein is a stable composition of an oxine-containing cell radiolabelling agent, said composition comprising an oxine-containing cell radiolabelling agent, a surfactant, a base, a buffering agent and a solvent. In some embodiments, the composition is a stable composition of ⁸⁹Zr-oxine, ⁶⁸Ga-oxine, ⁶⁴Cu-oxine, ¹¹¹In-oxine, ⁶⁷Ga-oxine or ⁵²Mn-oxine. In some embodiments, the composition is a stable composition of ⁸⁹Zr-oxine, ⁶⁸Ga-oxine, or ⁶⁴Cu-oxine. In preferred embodiments, the composition is a stable composition of ⁸⁹Zr-oxine. The stable composition of the oxine-containing cell radiolabelling agent is preferably prepared using methods as described herein.

In a further aspect of the invention, provided herein is a method of labelling cells, liposomes or extracellular vesicles with the oxine-containing cell radiolabelling agent, comprising the step of incubating cells, liposomes or extracellular vesicles with the stable composition as described herein comprising: an oxine-containing cell radiolabelling agent, a surfactant, a base, a buffering agent, and a solvent. In some embodiments, the composition is a stable composition of ⁸⁹Zr-oxine, ⁶⁸Ga-oxine, ⁶⁴Cu-oxine, ¹¹¹In-oxine, ⁶⁷Ga-oxine or ⁵²Mn-oxine. In some embodiments, the composition is a stable composition of ⁸⁹Zr-oxine, ⁶⁸Ga-oxine, or ⁶⁴Cu-oxine. In preferred embodiments, the composition is a stable composition of ⁸⁹Zr-oxine. In some embodiments the method further comprises (i.e. is preceded by) the ‘one step’ method for preparing oxine-containing cell radiolabelling agents described above.

In a further aspect of the invention, provided herein is a method of imaging comprising the steps of: incubating cells, liposomes or extracellular vesicles with the stable composition as described herein comprising: an oxine-containing cell radiolabelling agent, a surfactant, a base, a buffering agent, and a solvent; administering cells,liposomes or extracellular vesicles labelled with the oxine-containing cell radiolabelling agent as described herein to a subject and examining the subject using a suitable imaging technique, for example PET imaging or SPECT imaging. Preferably, the imaging technique is PET imaging. In some embodiments, the composition is a stable composition of ⁸⁹Zr-oxine, ⁶⁸Ga-oxine, ⁶⁴Cu-oxine, ¹¹¹In-oxine, ⁶⁷Ga-oxine or ⁵²Mn-oxine. In some embodiments, the composition is a stable composition of ⁸⁹Zr-oxine, ⁶⁸Ga-oxine, or ⁶⁴Cu-oxine. In preferred embodiments, the composition is a stable composition of ⁸⁹Zr-oxine. In some embodiments the method further comprises (i.e. is preceded by) the ‘one step’ method for preparing oxine-containing cell radiolabelling agents described above.

In a further aspect of the invention, provided herein is a formulation, kit or composition as described herein for use in imaging, i.e. medical imaging using a suitable imaging technique such as PET or SPECT. Preferably, the imaging technique is PET imaging.

In another aspect of the invention, provided herein is the use of a formulation, kit or composition as described herein for imaging, i.e. medical imaging using a suitable imaging technique such as PET or SPECT. Preferably, the imaging technique is PET imaging.

In a further aspect of the invention, provided herein is a method of diagnosing infection or inflammation comprising: incubating cells with the stable composition as described herein comprising: an oxine-containing cell radiolabelling agent, a surfactant, a base, a buffering agent and a solvent; administering cells labelled with the oxine-containing cell radiolabelling agent as described herein to a subject; examining the subject using a suitable imaging technique, for example PET imaging or SPECT imaging; locating sites of infection or inflammation. In some embodiments, the composition is a stable composition of ⁸⁹Zr-oxine, ⁶⁸Ga-oxine, ⁶⁴Cu-oxine, ¹¹¹In-oxine, ⁶⁷Ga-oxine or ⁵²Mn-oxine. In some embodiments, the composition is a stable composition of ⁸⁹Zr-oxine, ⁶⁸Ga-oxine, or ⁶⁴Cu-oxine. In preferred embodiments, the composition is a stable composition of ⁸⁹Zr-oxine.

In a further aspect of the invention, provided herein is a formulation, kit or composition as described herein for use in diagnosing infection or inflammation. In some embodiments the formulation, kit or composition is used to diagnose infection in bone, soft tissue, an organ or a lymph node. In some embodiments the formulation, kit or composition is used to diagnose infection or inflammation in a vascular graft. In some embodiments the formulation, kit or composition is used to diagnose infection or inflammation in a prosthetic joint.

In another aspect of the invention, provided herein is the use of a formulation, kit or composition as described herein for diagnosing infection or inflammation. In some embodiments the formulation, kit or composition is used to diagnose infection in bone, soft tissue, an organ or a lymph node. In some embodiments the formulation, kit or composition is used to diagnose infection or inflammation in a vascular graft. In some embodiments, the formulation, kit or composition is used to diagnose infection or inflammation in a prosthetic joint.

In a further aspect of the invention, provided herein is a method of diagnosing blood pooling or internal bleeding comprising: incubating cells with the stable composition as described herein comprising: an oxine-containing cell radiolabelling agent, a surfactant, a base, a buffering agent and a solvent; administering cells labelled with the oxine-containing cell radiolabelling agent as described herein to a subject; examining the subject using a suitable imaging technique, for example PET imaging or SPECT imaging; locating sites of blood pooling or internal bleeding. In some embodiments, the composition is a stable composition of ⁸⁹Zr-oxine, ⁶⁸Ga-oxine, ⁶⁴Cu-oxine, ¹¹¹In-oxine, ⁶⁷Ga-oxine or ⁵²Mn-oxine. In some embodiments, the composition is a stable composition of ⁸⁹Zr-oxine, ⁶⁸Ga-oxine, or ⁶⁴Cu-oxine. In preferred embodiments, the composition is a stable composition of ⁸⁹Zr-oxine. In other preferred embodiments, the composition is a stable composition of ⁶⁴Cu-oxine.

In another aspect of the invention, provided herein is a formulation, kit or composition as described herein for use in detecting blood pooling or internal bleeding. In some embodiments the formulation, kit or composition is used to diagnose gastrointestinal bleeding. In some embodiments the formulation, kit or composition is used to diagnose lower gastrointestinal bleeding.

In a further aspect of the invention, provided herein is the use of a formulation, kit or composition as described herein for detecting blood pooling or internal bleeding. In some embodiments the formulation, kit or composition is used to diagnose gastrointestinal bleeding. In some embodiments the formulation, kit or composition is used to diagnose lower gastrointestinal bleeding.

In another aspect of the invention, provided herein is a method of cell therapy comprising: incubating cells with the stable composition as described herein comprising: an oxine-containing cell radiolabelling agent, a surfactant, a base, a buffering agent, and a solvent; administering cells labelled with the oxine-containing cell radiolabelling agent as described herein to a subject; examining the subject using a suitable imaging technique, for example PET imaging or SPECT imaging; locating the administered cells. In some embodiments the cells are CAR-T cells. In some embodiments the cells are gamma-delta T cells.

In some embodiments the cell therapy treatment is CAR-T treatment. In some embodiments the cell therapy treatment is gamma-delta T cell treatment. In some embodiments the cells are stem cells. In some preferred embodiments the cells are mesenchymal stem cells. In some other preferred embodiments the cells are haematopoietic stem cells. In some embodiments the cell therapy treatment is a stem cell treatment. In some embodiments the cell therapy treatment is mesenchymal stem cell therapy. In some embodiments the cell therapy treatment is haematopoietic stem cell transplantation.

In a further aspect of the invention, provided herein is a formulation, kit or composition as described herein for use in cell therapy treatments. In some embodiments the formulation, kit or composition can be used in stem cell treatments. In some embodiments the formulation, kit or composition can be used in mesenchymal stem cell therapy. In some embodiments the formulation, kit or composition can be used in haematopoietic stem cell transplantation. In some embodiments the formulation, kit or composition can be used in T cell therapy treatments. In some embodiments the formulation, kit or composition can be used in CAR-T cell treatments. In some embodiments the formulation, kit or composition can be used in gamma-delta T cell treatments.

In a further aspect of the invention, provided herein is the use of a formulation, kit or composition as described herein for cell therapy. In some embodiments the formulation, kit or composition can be used in stem cell treatments. In some embodiments the formulation, kit or composition can be used in mesenchymal stem cell therapy. In some embodiments the formulation, kit or composition can be used in haematopoietic stem cell transplantation. In some embodiments the formulation, kit or composition can be used in T cell therapy treatments. In some embodiments the formulation, kit or composition can be used in CAR-T cell treatments. In some embodiments the formulation, kit or composition can be used in gamma-delta T cell treatments.

In a further aspect of the invention, provided herein is an oxine-containing cell radiolabelling agent prepared by a method as described herein.

In a further aspect of the invention, provided herein is a radiolabelled cell prepared by a method as described herein. Also provided is a radiolabelled liposome prepared by a method as described herein. Also provided is a radiolabelled extracellular vesicle prepared by a method as described herein.

In another aspect of the invention, provided herein is a method of nanomedicine delivery comprising: incubating liposomes or extracellular vesicles with the stable composition as described herein comprising: an oxine-containing cell radiolabelling agent, a surfactant, a base, a buffering agent and a solvent; administering liposomes or extracellular vesicles labelled with the oxine-containing cell radiolabelling agent as described herein to a subject; examining the subject using a suitable imaging technique, for example PET imaging or SPECT imaging. In some embodiments the liposome or extracellular vesicle encapsulates a cargo. In some embodiments the liposome encapsulates a drug.

In a further aspect of the invention, provided herein is a formulation, kit or composition as described herein for use in nanomedicine delivery.

In a further aspect of the invention, provided herein is the use of a formulation, kit or composition as described herein for nanomedicine delivery.

In a further aspect of the invention, provided herein is a method of diagnosing cancer comprising: incubating cells, liposomes or extracellular vesicles with the stable composition as described herein comprising: an oxine-containing cell radiolabelling agent, a surfactant, a base, a buffering agent and a solvent; administering cells, liposomes or extracellular vesicles labelled with the oxine-containing cell radiolabelling agent as described herein to a subject; examining the subject using a suitable imaging technique, for example PET imaging or SPECT imaging; locating sites of cancer.

In a further aspect, provided herein is a formulation, kit or composition for use in diagnosing cancer.

In a further aspect, provided herein is the use of a formulation, kit or composition as described herein for diagnosing cancer.

In another aspect of the invention, provided herein is an imaging method comprising the steps of: incubating cells, liposomes or extracellular vesicles with the stable composition as described herein comprising: an oxine-containing cell radiolabelling agent, a surfactant, a base, a buffering agent and a solvent; and examining the cells, liposomes or extracellular vesicles using a suitable imaging technique, for example PET imaging or SPECT imaging. Preferably, the imaging technique is PET imaging. In some embodiments, the composition is a stable composition of ⁸⁹Zr-oxine, ⁶⁸Ga-oxine, ⁶⁴Cu-oxine, ¹¹¹In-oxine, ⁶⁷Ga-oxine or ⁵²Mn-oxine. In some embodiments, the composition is a stable composition of ⁸⁹Zr-oxine, ⁶⁸Ga-oxine, or ⁶⁴Cu-oxine. In preferred embodiments, the composition is a stable composition of ⁸⁹Zr-oxine. In some embodiments the method further comprises (i.e. is preceded by) the ‘one step’ method for preparing oxine-containing cell radiolabelling agents described above. In some embodiments the imaging method is performed in vitro.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures:

FIG. 1. Radiochromatogram showing the formation of ⁸⁹Zr-oxine as streaking between the origin and the solvent front (Rt=1). ⁸⁹Zr-oxalate was added to 100 µL of the formulation of the invention, spotted on ITLC-SG paper, left to run in 100% ethyl acetate and analysed on a linear TLC reader equipped with a p+ probe.

FIG. 2. Radiochromatogram showing the formation of ⁸⁹Zr-oxine as the peak appearing at the solvent front (Rf=1). ⁸⁹Zr-oxalate was added to 100 µL of the formulation of the invention, spotted on Whatman paper, left to run in 100% ethyl acetate and analysed on a linear TLC reader equipped with a p+ probe.

FIG. 3. Radiochromatogram of ⁸⁹Zr oxalate spotted on Whatman no. 1 paper, left to run in 100% ethyl acetate and analysed on a linear TLC reader equipped with a p+ probe.

FIG. 4. Radiochromatogram showing the formation of oxine complexes of ⁶⁴Cu as the peak appearing above the origin. An acidic solution of ⁶⁴Cu (approx. 1 M HCI) was added to 100 µL of the formulation of the invention and spotted on Whatman no.1 paper, left to run in 100% ethyl acetate and analysed on a linear TLC reader equipped with a p+ probe.

FIG. 5. Radiochromatogram showing the formation of oxine complexes of ⁶⁸Ga as the peak appearing above the origin. An acidic solution of ⁶⁸Ga (0.1 M HCI) was added to 100 µL of formulation of the invention, spotted on Whatman no.1 paper, left to run in 100% ethyl acetate and analysed on a linear TLC reader equipped with a p+ probe.

FIG. 6. Radiochromatogram of acidic solutions of ⁶⁴Cu and ⁶⁸Ga without the formulation of the invention spotted on Whatman no. 1 paper, left to run in 100% ethyl acetate and analysed on a linear TLC reader equipped with a p+ probe.

FIG. 7. Formation speed and radiochemical yield of ⁸⁹Zr-oxine as a function of oxalic acid content and ⁸⁹Zr decay. ⁸⁹Zr in 1 M oxalic acid (1.1-1.5 MBq/µL on day 0, approximately 4-5 days after production in the cyclotron) was added to 20 µL aliquots of kit formulation on day of reception (day 0) and 4 subsequent days. To keep total ⁸⁹Zr activity constant, increasing volumes of ⁸⁹Zr solution were added on each day, resulting in increasing concentrations of oxalate ions in the final product. Radiochemical yield was determined by radioTLC at 1, 5, 10 and 15 min after addition of ⁸⁹Zr.

FIG. 8. Comparison of the speed of formation of ⁸⁹Zr-oxine complexes with formulations containing NaOH (dots) or NaHCO₃ (squares) as base. ⁸⁹Zr-oxalate was added to 100 µL of the formulation of the invention containing NaOH or NaHCO₃ and spotted after 1, 5, 10 and 15 min on Whatman no.1 paper. The yield is calculated as the area under the curve (AUC) of the peak at Rf=1 as a percentage of the sum of AUCs of the peaks at Rf=1 and Rf=0 (see FIG. 1). Mean±SD of n=2-3 separate experiments.

FIG. 9. Recovery of ⁸⁹Zr-oxine complexes from the reaction vial over time. The figure compares formulations containing 8-hydroxyquinoline, HEPES and NaOH only (dots), and additional 5% EtOH (squares) or 1 mg/mL polysorbate 80 (diamonds). ⁸⁹Zr-oxalate was added to 100 µL of the formulation of the invention in a glass vial and left at room temperature for up to 1 week. 10 µL aliquots were removed from the glass vial immediately after addition (0 min) and after 15 min, 30 min, 1, 2, 24, 48, 72 and 168 h, then gamma-counted at the end of the experiment. The recovered activity is expressed as the activity in the aliquot at the indicated time as a percentage of the activity sampled immediately after addition of ⁸⁹Zr to the-formulation of the invention. Mean±SD, N=3.

FIG. 10. Recovery of ⁸⁹Zr-oxine complexes from the reaction vial over time as a function of the amount of polysorbate 80. The figure compares formulations containing 8-hydroxyquinoline, HEPES, NaOH and varying amounts of polysorbate 80. ⁸⁹Zr-oxalate was added to 100 µL of 8-hydroxyquinoline solutions in a glass vial and left at room temperature for up to 1 week. 10 µL aliquots were removed from the glass vial immediately after addition (0 min) and after 15 min, 30 min, 1, 2, 24, 48, 72 and 168 h, then gamma-counted at the end of the experiment. The recovered activity is expressed as the activity in the aliquot at the indicated time as a percentage of the activity sampled immediately after addition of ⁸⁹Zr to the formulation of the invention. Mean±SD, N=3.

FIG. 11. Stability of the ⁸⁹Zr-complex over time. ⁸⁹Zr-oxalate was added to 100 µL of the formulation of the invention (squares) or 100 µL of the formulation of the invention + 900 µL H₂O (“diluted” kit, dots) in a glass vial and left at room temperature for up to 1 week. Aliquots were removed after 24, 48, 72 and 168 h and analysed by radioTLC on Whatman no.1 paper.

FIG. 12. Radiochromatogram showing formation of ⁸⁹Zr-oxine from ⁸⁹Zr in chloride form. ⁸⁹Zr-oxalate was loaded onto a QMA ion-exchange cartridge, washed with water, and the cartridge was eluted with 500 µL 1 M HCI to obtain ⁸⁹Zr chloride. ⁸⁹Zr chloride was added to 100 µL of the formulation of the invention and analysed by radioTLC on Whatman no.1 paper.

FIG. 13. Radiochromatogram showing formation of ⁸⁹Zr-oxine from ⁸⁹Zr in oxalate form. ⁸⁹Zr-oxalate was added to 100 µL of the formulation of the invention and analysed by radioTLC on Whatman no.1 paper.

FIG. 14. WBC radiolabelling with ⁸⁹Zr-oxine and ¹¹¹In-oxine. WBC labelling efficiency. Mean ± SD of n=10 per group. ****p<0.0001 (Student’s two-tailed paired t-test).

FIG. 15. WBC radiolabelling with ⁸⁹Zr-oxine and ¹¹¹In-oxine. Retention of ⁸⁹Zr-oxine and ¹¹¹In-oxine. Mean ± SD of n=10 per group. ****p<0.0001 (Student’s two-tailed paired t-test).

FIG. 16. WBC radiolabelling with ⁸⁹Zr-oxine and ¹¹¹In-oxine. Percentage of viable ⁸⁹Zr- and ¹¹¹n-labelled WBC after 4 h and 24 h, measured using the Trypan blue assay. Mean ± SD of n=6 per group, Student’s two-tailed paired t-test. ns: not significant (p≥0.05).

FIG. 17. Chemotaxis of radiolabelled WBC. Cells were incubated with ⁸⁹Zr-oxine, ¹¹¹In-oxine, or vehicle only. The chemotaxis index is the number of cells migrating towards 10 nM fMLP divided by the number of cells migrating towards vehicle, averaged from triplicates for each sample. Each point represents one donor. Bars represent the mean ± SD of n = 9, 10 and 10 individual samples. p≥0.05.

FIG. 18. Relative activity per cell of ⁸⁹Zr in WBC populations after labelling of mixed WBC with ⁸⁹Zr-oxine. Radiolabelled WBC were sorted by FACS and gamma-counted. Each point represents cells from an individual donor.

FIG. 19. Determination of optimal TLC strip length for QC of ⁸⁹Zr-oxine. Representative radioTLC chromatograms of ⁸⁹Zr-oxine on Whatman no.1 paper, with 100% ethyl acetate as mobile phase.

FIG. 20. Use of the kit formulation to form lipophilic oxine complexes of ⁶⁴Cu, ⁶⁸Ga and ¹¹¹In. Radionuclides were added in the typical form in which they are supplied from cyclotron target extraction or generator elution. To 100 µL of the formulation of the invention, ⁶⁴Cu in 1-2 M HCl, ⁶⁸Ga in 0.1 M HCl or ¹¹¹In in 0.1 M HCI were added and left for 10 min at RT. RadioTLCs were performed on Whatman no.1 paper as described previously.

FIG. 21. Use of the kit formulation to radiolabel cells for imaging purposes. Representative PET/CT images (coronal view) of immunocompromised mice (NSG) bearing human breast cancer tumours (HCC1954, implanted on day 0) and injected intravenously (day 28) with human gammadelta T cells radiolabelled with ⁸⁹Zr-oxine. Images were acquired 48 h (on day 30) after administration of radiolabelled cells and show increased uptake of gammadelta T cells in tumours in mice that were additionally treated with PEGylated liposomal alendronate (PLA, right), compared to untreated mice.

FIG. 22. Representative immunohistochemistry images of HCC1954 tumour sections stained for human CD3 expression (surrogate marker for human gammadelta T cells; brown spots) on day 63/64 after tumour implantation, showing the persistence of intravenously injected, radiolabelled gammadelta T cells well beyond the decay of ⁸⁹Zr.

FIG. 23. Use of the kit formulation to radiolabel extracellular vesicles (EVs) for imaging purposes. Radiolabelling efficiency of EVs derived from various cancer cell lines (B16-F10.GFP, MDA-MB-231.CD63-GFP, PANC1 and 4T1) and from 2D and 3D cultures of mesenchymal stem cells (MSC), showing higher labelling efficiency with ⁸⁹Zr-oxine compared to unchelated ⁸⁹Zr.

FIG. 24. Representative PET/CT images of ⁸⁹Zr-labelled PANC1-derived EVs after intravenous administration in a C57/BL6 mouse, showing uptake in the liver, spleen and lymph nodes (LNs, arrowheads).

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

The present invention provides methods, kits and formulations for the preparation of oxine-containing cell radiolabelling agents, including but not limited to ⁸⁹Zr-oxine, ⁶⁸Ga-oxine, ⁶⁴Cu-oxine, ¹¹¹In-oxine, ⁶⁷Ga-oxine and ⁵²Mn-oxine. Also provided is a stabilised composition of such an oxine-containing cell radiolabelling agent, i.e. which is obtained by or obtainable by the methods described herein. An oxine formulation as described herein, for use in the methods described herein, comprises: oxine; a surfactant; a base; a buffering agent, and optionally a solvent. A stabilised composition as described herein comprises an oxine-containing cell radiolabelling agent; a surfactant; a base; a buffering agent, and a solvent.

The term ‘oxine’ as used herein refers to 8-hydroxyquinoline.

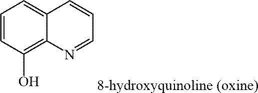

The term ‘oxine’ is also used herein to denote the corresponding moiety in any complex formed by 8-hydroxyquinoline or its conjugate base with another chemical species e.g. an oxinate. In some embodiments the chemical species is a radioactive metal. For example, ⁸⁹Zr-oxine, ⁶⁸Ga-oxine, ⁶⁴Cu-oxine, ¹¹¹In-oxine, ⁶⁷Ga-oxine or ⁵²Mn-oxine refer to any complex of 8-hydroxyquinoline with ⁸⁹Zr, ⁶⁸Ga, ⁶⁴Cu, ¹¹¹In, ⁶⁷Ga, ⁵²Mn.

In some embodiments ⁸⁹Zr-oxine is the following complex:

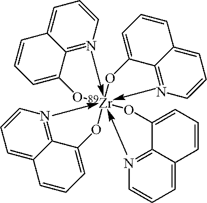

In some embodiments ⁶⁸Ga-oxine is the following complex:

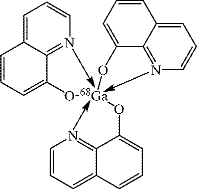

In some embodiments ⁶⁴Cu-oxine is the following complex:

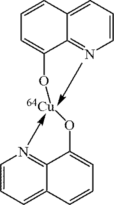

In some embodiments ¹¹¹In-oxine is the following complex:

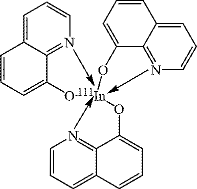

In some embodiments ⁶⁷Ga-oxine is the following complex:

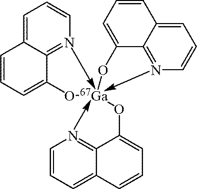

In some embodiments ⁵²Mn-oxine is the following complex:

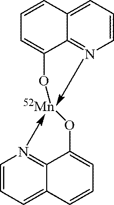

In some embodiments the formulation contains between 0.01 mg and 1 mg of oxine. In some embodiments the formulation contains between 0.01 mg and 0.1 mg of oxine. In some embodiments the formulation contains between 0.01 mg and 0.08 mg of oxine. In a preferred embodiment the formulation contains 0.05 mg of oxine.

Preferably the formulation described herein contains between 1 × 10⁻⁶ mmol and 0.07 mmol of oxine. In some embodiments the formulation contains between 1 × 10⁻⁵ mmol and 0.007 mmol of oxine. In some embodiments the formulation contains between 1 × 10⁻⁴ mmol and 0.0009 mmol of oxine. In some embodiments the formulation contains between 1 × 10⁻⁴ mmol and 0.0007 mmol of oxine. In some embodiments the formulation contains between 0.0001 mmol and 0.0006 mmol of oxine. In a preferred embodiment the formulation contains 0.0003 mmol of oxine.

The formulations and compositions described herein include a surfactant. Without wishing to be bound by theory, it is thought to be advantageous to include a surfactant, to prevent the adhesion of newly formed oxine-containing cell radiolabelling agent to the surface of the vessel in which it is formed. In some embodiments, the surfactant used can be a non-ionic, anionic, or cationic surfactant, or a combination thereof. Examples of non-ionic surfactants include alkyl polyglycoside, cetomacrogol 1000, cetostearyl alcohol, cetyl alcohol, cocamide DEA, cocamide MEA, decyl glucoside, decyl polyglucose, glycerol monostearate, isoceteth-20, lauryl glucoside, maltosides, monolaurin, mycosubtilin, narrow-range ethoxylate, nonoxynol-9, nonoxynols, octaethylene glycol monododecyl ether, N-octyl beta-D-thioglucopyranoside, octyl glucoside, oleyl alcohol, PEG-10 sunflower glycerides, pentaethylene glycol monododecyl ether, polidocanol, poloxamer, poloxamer 407, polyethoxylated tallow amine, polyglycerol polyricinoleate, polysorbate, polysorbate 20, polysorbate 80, sorbitan, sorbitan monolaurate, sorbitan monostearate, sorbitan tristearate, stearyl alcohol, surfactin, tween 80, ethanol or dimethyl sulfoxide.

In some embodiments the non-ionic surfactant may preferably be selected from alkyl polyglycoside, cetomacrogol 1000, cetostearyl alcohol, cetyl alcohol, cocamide DEA, cocamide MEA, decyl glucoside, decyl polyglucose, glycerol monostearate, isoceteth-20, lauryl glucoside, maltosides, monolaurin, mycosubtilin, narrow-range ethoxylate, nonoxynol-9, nonoxynols, octaethylene glycol monododecyl ether, N-octyl beta-D-thioglucopyranoside, octyl glucoside, oleyl alcohol, PEG-1 0 sunflower glycerides, pentaethylene glycol monododecyl ether, polidocanol, polyethoxylated tallow amine, polyglycerol polyricinoleate, polysorbate, polysorbate 20, polysorbate 80, sorbitan, sorbitan monolaurate, sorbitan monostearate, sorbitan tristearate, stearyl alcohol, surfactin, tween 80, ethanol or dimethyl sulfoxide.

Examples of anionic surfactants include 2-acrylamido-2-methylpropane sulfonic acid, alkylbenzene sulfonates, ammonium lauryl sulfate, ammonium perfluorononanoate, chlorosulfolipid, dioctyl sodium sulfosuccinate, disodium cocoamphodiacetate, magnesium laureth sulfate, perfluorobutanesulfonate, perfluorononanoic acid, perfluorooctanesulfonate, perfluorooctanoate, potassium lauryl sulfate, sodium alkyl sulfate, sodium dodecyl sulfate, sodium laurate, sodium laureth sulfate, sodium lauryl ether sulfate, sodium lauroyl sarcosinate, sodium myreth sulfate, sodium nonanoyloxybenzenesulfonate, sodium pareth sulfate, sodium stearate, sodium sulfosuccinate esters.

Examples of cationic surfactants include behentrimonium chloride, benzalkonium chloride, benzethonium chloride, benzododecinium bromide, bronidox, carbethopendecinium bromide, cetalkonium chloride cetrimonium bromide, cetrimonium chloride, cetylpyridinium chloride, didecyldimethylammonium chloride, dimethyldioctadecylammonium bromide, dimethyldioctadecylammonium chloride, dioleoyl-3-trimethylammonium propane, domiphen bromide, lauryl methyl gluceth-10 hydroxypropyl dimonium chloride, octenidine dihydrochloride, olaflur, N-Oleyl-1,3-propanediamine, pahutoxin, stearalkonium chloride, tetramethylammonium hydroxide, thonzonium bromide.

The surfactant may also be another surfactant commonly known in the art.

Preferably the surfactant is selected from the group of polysorbate 80, ethanol, dimethyl sulfoxide or a combination thereof. More preferably the surfactant is polysorbate 80.

In some embodiments there is between 0.1 and 200 mg of surfactant per mg of the oxine in the formulation. In some embodiments there is between 0.5 and 150 mg of surfactant per mg of the oxine in the formulation. In some embodiments there is between 1 and 100 mg of surfactant per mg of the oxine in the formulation. In some embodiments there is between 1.5 and 50 mg of surfactant per mg of the oxine in the formulation. In some embodiments there is between 1.5 and 3 mg of surfactant per mg of the oxine in the formulation. In some embodiments there is about 2 mg of surfactant per mg of the oxine in the formulation.

In some embodiments there is between 0.01 and 20 mmol of surfactant per mmol of the oxine in the formulation. In some embodiments there is between 0.05 and 15 mmol of surfactant per mmol of the oxine in the formulation. In some embodiments there is between 0.15 and 15 mmol of surfactant per mmol of the oxine in the formulation. In some embodiments there is between 0.15 and 5 mmol of surfactant per mmol of the oxine in the formulation. In some embodiments there is between 0.15 and 0.30 mmol of surfactant per mmol of the oxine in the formulation. In some embodiments there is about 0.22 mmol of surfactant per mmol of the oxine in the formulation.

Advantageously, by optimising the surfactant content in the formulation the newly formed oxine-containing cell radiolabelling agent remains stable in the resultant composition for at least 1 day. In some embodiments the newly formed oxine-containing cell radiolabelling agent remains stable in the composition for at least 3 days. In some embodiments the newly formed oxine-containing cell radiolabelling agent remains stable in the composition for at least 7 days.

In some embodiments, ready-to-use oxine-containing cell radiolabelling agent can be provided in the stable composition described herein i.e. such that the oxine-containing cell radiolabelling agent can be used without further processing. In some embodiments ready-to-use oxine-containing cell radiolabelling agent can be shipped in the stable composition i.e. such that the oxine-containing cell radiolabelling agent can be used without further processing.

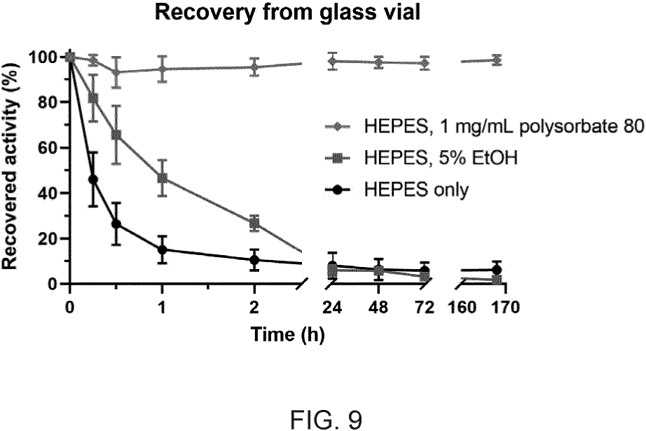

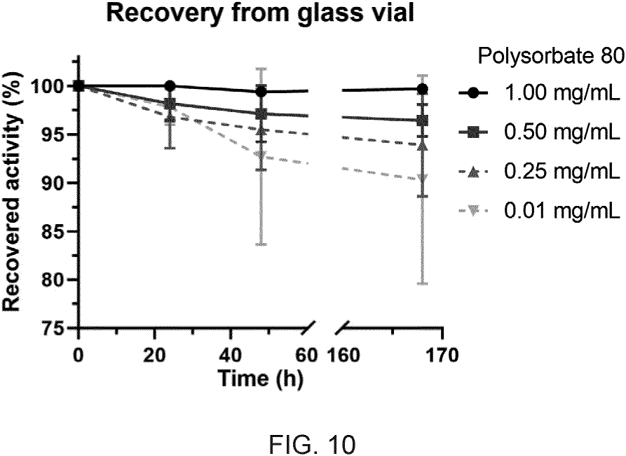

The stability of the compositions described herein can be assessed, for example, in terms of their recovered activity. The recovered activity is expressed as the percentage of activity (i.e. radioactivity, as assessed by gamma-counting) in a sample at the time of measurement, relative to the activity sampled immediately after addition of the radioactive metal containing solution to the oxine formulation described herein. See FIGS. 9 and 10, for example. The recovered activity can be reduced for example by adherence of the oxine-containing cell radiolabelling agents as described herein to a surface.

In some embodiments, after the addition of the radioactive metal containing solution to the formulation the recovered activity remains above 50% for at least 1 day. In some embodiments, the recovered activity remains above 50% for at least 3 days. In some embodiments, the recovered activity remains above 50% for at least 7 days. In some embodiments, after the addition of the radioactive metal containing solution to the formulation the recovered activity remains above 60% for at least 1 day. In some embodiments, the recovered activity remains above 60% for at least 3 days. In some embodiments, the recovered activity remains above 60% for at least 7 days. In some embodiments, after the addition of the radioactive metal containing solution to the formulation the recovered activity remains above 70% for at least 1 day. In some embodiments, the recovered activity remains above 70% for at least 3 days. In some embodiments, the recovered activity remains above 70% for at least 7 days. In some embodiments, after the addition of the radioactive metal containing solution to the formulation the recovered activity remains above 80% for at least 1 day. In some embodiments, the recovered activity remains above 80% for at least 3 days. In some embodiments, the recovered activity remains above 80% for at least 7 days.

The formulations and compositions described herein include a base. Without wishing to be bound by theory, it may be advantageous to include a base in the oxine formulation to neutralise the solution in which the radioactive metal is provided.

In some embodiments, the base is a water-soluble base. In some embodiments, the base preferably is or comprises an alkali metal hydroxide, carbonate or bicarbonate, or a mixture thereof. In some embodiments the base in the formulation preferably is or comprises an alkali metal hydroxide. In some embodiments the base in the formulation preferably is or comprises sodium hydroxide. Other bases common in the art may also be used.

In some embodiments there is between 1 and 250 mg of base per mg of the oxine in the formulation. In some embodiments there is between 10 and 150 mg of base per mg of the oxine in the formulation. In some embodiments there is between 10 and 100 mg of base per mg of the oxine in the formulation. In some embodiments there is between 20 and 60 mg of base per mg of the oxine in the formulation. In some embodiments there is between 30 and 50 mg of base per mg of the oxine in the formulation. In some embodiments there is between 35 and 50 mg of base per mg of the oxine in the formulation. In some embodiments there is about 42 mg of base per mg of the oxine in the formulation.

In some embodiments there is between 50 and 800 mmol of base per mmol of the oxine in the formulation. In some embodiments there is between 60 and 700 mmol of base per mmol of the oxine in the formulation. In some embodiments there is between 100 and 600 mmol of base per mmol of the oxine in the formulation. In some embodiments there is between 100 and 400 mmol of base per mmol of the oxine in the formulation. In some embodiments there is between 100 and 200 mmol of base per mmol of the oxine in the formulation. In some embodiments there is between 125 and 175 mmol of base per mmol of the oxine in the formulation. In some embodiments there is about 153 mmol of base per mmol of oxine in the formulation.

In some embodiments, sodium hydroxide is used as the base in the formulation. This may have the advantage that a high yield of oxine-containing cell radiolabelling agent can be formed quickly. The yield of oxine-containing cell radiolabelling agent produced by the methods described herein can be calculated using Thin Layer Chromatography (TLC). The yield of oxine-containing cell radiolabelling agent produced by the methods described herein can be calculated by finding the area under the curve (AUC) of the radioactive metal peak of the TLC as a percentage of the sum of AUCs of the oxine-containing cell radiolabelling agent peak and radioactive metal peak of the TLC. As used herein, the term ‘yield’ means the percentage of the radioactive metal which is successfully complexed to oxine.

In some embodiments, the yield of the oxine-containing cell radiolabelling agent is above 40%. In some embodiments the yield of the oxine-containing cell radiolabelling agent is above 50%. In some embodiments the yield of the oxine-containing cell radiolabelling agent is above 60%. In some embodiments the yield of the oxine-containing cell radiolabelling agent is above 70%. In some embodiments the yield of the oxine-containing cell radiolabelling agent is above 80%.

In some embodiments a yield of above 40% is achieved within 20 minutes of addition of the radioactive metal to the formulation. In some embodiments a yield of above 40% is achieved within 10 minutes. In some embodiments a yield of above 40% is achieved within 5 minutes. In some embodiments a yield of above 50% is achieved within 20 minutes. In some embodiments a yield of above 50% is achieved within 10 minutes. In some embodiments a yield of above 50% is achieved within 5 minutes. In some embodiments a yield of above 60% is achieved within 20 minutes. In some embodiments a yield of above 60% is achieved within 10 minutes. In some embodiments a yield of above 60% is achieved within 5 minutes. In some embodiments a yield of above 70% is achieved within 20 minutes. In some embodiments a yield of above 70% is achieved within 10 minutes. In some embodiments a yield of above 70% is achieved within 5 minutes. In some embodiments a yield of above 80% is achieved within 20 minutes. In some embodiments a yield of above 80% is achieved within 10 minutes. In some embodiments a yield of above 80% is achieved within 5 minutes. In a preferred embodiment a yield of above 80% is achieved within 5 minutes of addition of the radioactive metal containing solution to the formulation.

The formulations and compositions described herein include a buffering agent. The presence of a buffering agent in the formulation may preferably keep the pH of the formulation within a range suitable for the formation and maintained stability of the oxine-containing cell radiolabelling agent.

In some embodiments, the buffering agent in the formulation is selected from the group of Tris (tris(hydroxymethyl)aminomethane), HEPES (4-(2-hydroxyethyl)-1-piperazineethanesulfonic acid), MOPS (3-(N-morpholino)propanesulfonic acid), PIPES (piperazine-N,N′-bis(2-ethanesulfonic acid)), ACES (2-(carbamoylmethylamino)ethanesulfonic acid), MOPSO (2-hydroxy-3-morpholin-4-ylpropane-1-sulfonic acid), Cholamine chloride hydrochloride (2-aminoethyl(trimethyl)azanium;chloride;hydrochloride), HEPPS (3-[4-(2-Hydroxyethyl)piperazin-1-yl]propane-1-sulfonic acid), glycinamide (2-Aminoacetamide), Glycylglycine (2-[(2-Aminoacetyl)amino]acetic acid), HEPBS (N-(2-Hydroxyethyl)piperazine-N′-(4-butanesulfonic acid)). Other buffers commonly known in the art may also be used.

As will be understood by those skilled in the art, most buffering agents in aqueous solution consist of a weak acid and its conjugate base. For the avoidance of doubt, the ‘base’ included in the formulations of the invention (as described above) is different from any conjugate base which may be present as a result of the buffering agent.

In some embodiments the buffering agent in the formulation is or comprises HEPES. In some embodiments there is between 100 and 5000 mg of buffering agent per mg of the oxine in the formulation. In some embodiments there is between 200 and 1500 mg of buffering agent per mg of the oxine in the formulation. In some embodiments there is between 300 and 1000 mg of buffering agent per mg of the oxine in the formulation. In some embodiments there is between 300 and 800 mg of buffering agent per mg of the oxine in the formulation. In some embodiments there is between 400 and 500 mg of buffering agent per mg of the oxine in the formulation. In some embodiments there is about 477 mg of buffering agent per mg of the oxine in the formulation.

In some embodiments there are between 50 and 1500 mmol of buffering agent per mmol of the oxine in the formulation. In some embodiments there are between 100 and 1500 mmol of buffering agent per mmol of the oxine in the formulation. In some embodiments there are between 100 and 500 mmol of buffering agent per mmol of the oxine in the formulation. In some embodiments there are between 150 and 400 mmol of buffering agent per mmol of the oxine in the formulation. In some embodiments there are between 200 and 300 mmol of buffering agent per mmol of the oxine in the formulation. In some embodiments there is about 291 mmol of buffering agent per mmol of the oxine in the formulation.

In some embodiments the buffering agent maintains the pH of the oxine formulation described herein at a value between 6.5 and 8. In some embodiments the buffering agent maintains the pH of the formulation at a value between 7 and 8. In a preferred embodiment the buffering agent maintains the pH of the formulation at a value between 7.5 and 8. In a preferred embodiment the buffering agent maintains the pH of the formulation at a value between 7.8 and 8.

In some embodiments the oxine formulation described herein further comprises a solvent. In general, provision of the oxine formulations described herein would involve mixing / dissolving the relevant components in a suitable solvent, as would be understood by the person skilled in the art. In some embodiments the formulation comprises between 0.1 mL and 100 mL of solvent per mg of the oxine in the formulation. In some embodiments the formulation comprises between 0.5 mL and 50 mL of solvent per mg of the oxine in the formulation. In some embodiments the formulation comprises between 1 mL and 30 mL of solvent per mg of the oxine in the formulation. In some embodiments the formulation comprises between 2 mL and 20 mL of solvent per mg of the oxine in the formulation. In some embodiments the formulation comprises about 2 mL of solvent per mg of the oxine in the formulation.

In some embodiments the formulation comprises between 1 mL and 1000 mL of solvent per 0.1 mmol of the oxine in the formulation. In some embodiments the formulation comprises between 5 mL and 500 mL of solvent per 0.1 mmol of the oxine in the formulation. In some embodiments the formulation comprises between 10 mL and 300 mL of solvent per 0.1 mmol of the oxine in the formulation. In some embodiments the formulation comprises between 20 mL and 300 mL of solvent per 0.1 mmol of the oxine in the formulation. In some embodiments the formulation comprises about 29 mL of solvent per 0.1 mmol of the oxine in the formulation.

In some embodiments of the formulation or composition described herein the base is an alkali metal hydroxide and the buffering agent is selected from the group of Tris (tris(hydroxymethyl)aminomethane), HEPES (4-(2-hydroxyethyl)-1-piperazineethanesulfonic acid), MOPS (3-(N-morpholino)propanesulfonic acid), PIPES (piperazine-N,N′-bis(2-ethanesulfonic acid)), ACES (2-(carbamoylmethylamino)ethanesulfonic acid), MOPSO (2-hydroxy-3-morpholin-4-ylpropane-1-sulfonic acid), Cholamine chloride hydrochloride (2-aminoethyl(trimethyl)azanium;chloride;hydrochloride), HEPPS (3-[4-(2-Hydroxyethyl)piperazin-1-yl]propane-1-sulfonic acid), glycinamide (2-Aminoacetamide), Glycylglycine (2-[(2-Aminoacetyl)amino]acetic acid), HEPBS (N-(2-Hydroxyethyl)piperazine-N′-(4-butanesulfonic acid)).

In some preferred embodiments of the formulation or composition described herein the base is sodium hydroxide and the buffer is HEPES.

In some embodiments of the formulation or composition described herein the base is an alkali metal hydroxide, the buffering agent is selected from the group of Tris (tris(hydroxymethyl)aminomethane), HEPES (4-(2-hydroxyethyl)-1-piperazineethanesulfonic acid), MOPS (3-(N-morpholino)propanesulfonic acid), PIPES (piperazine-N,N′-bis(2-ethanesulfonic acid)), ACES (2-(carbamoylmethylamino)ethanesulfonic acid), MOPSO (2-hydroxy-3-morpholin-4-ylpropane-1-sulfonic acid), Cholamine chloride hydrochloride (2-aminoethyl(trimethyl)azanium;chloride;hydrochloride), HEPPS (3-[4-(2-Hydroxyethyl)piperazin-1-yl]propane-1-sulfonic acid), glycinamide (2-Aminoacetamide), Glycylglycine (2-[(2-Aminoacetyl)amino]acetic acid), HEPBS (N-(2-Hydroxyethyl)piperazine-N′-(4-butanesulfonic acid)) and the surfactant is selected from the group of polysorbate 80, ethanol, dimethyl sulfoxide or a combination thereof.

In some preferred embodiments of the formulation or composition described herein the base is sodium hydroxide, the buffer is HEPES and the surfactant is polysorbate 80.

In some embodiments of the formulation or composition described herein there is 0.15 and 0.30 mmol of surfactant, between 60 and 700 mmol of base and between 100 and 500 mmol of buffering agent per mmol of the oxine in the formulation or composition described herein.

In other embodiments, however, oxine formulations described herein may be free of solvent (e.g. a freeze-dried formulation). For use in the methods described herein, such an oxine formulation is preferably diluted or reconstituted in a suitable solvent. Accordingly, the resultant stable compositions of oxine-containing cell radiolabelling agents, as described herein generally comprise a solvent.

In some embodiments the volume of the formulation or composition is between 0.01 mL and 10 mL. In some embodiments the volume of the formulation or composition is between 0.05 mL and 5 mL. In some embodiments the volume of the formulation or composition is between 0.1 mL and 1 mL. Preferably, the volume of the formulation or composition is 0.1 mL.

Advantageously, in some embodiments the oxine-containing cell radiolabelling agent is stable in the composition of the invention for at least 1 days. In some embodiments the oxine-containing cell radiolabelling agent is stable in the composition of the invention for at least 2 days. In some embodiments the oxine-containing cell radiolabelling agent is stable in the composition of the invention for at least 3 days. In some embodiments the oxine-containing cell radiolabelling agent is stable in the composition of the invention for at least 4 days. In some embodiments the oxine-containing cell radiolabelling agent is stable in the composition of the invention for at least 5 days. In some embodiments the oxine-containing cell radiolabelling agent is stable in the composition of the invention for at least 6 days. In some embodiments the oxine-containing cell radiolabelling agent is stable in the composition of the invention for at least 7 days.

Possible solvents include 1, 4-dioxane, tetrahydrofuran, acetone, dimethylformamide, acetonitrile, dimethyl sulfoxide, ethanol, methanol, and mixtures thereof. In some embodiments the solvent is selected from the group of dimethyl sulfoxide or water and mixtures thereof. In some embodiments the solvent is water. It may be preferable to use water as a solvent, since the use of organic solvents is discouraged during the preparation of radiopharmaceuticals. A particular benefit of the invention is that the oxine-containing cell radiolabelling agents of the invention can be prepared using only water as the solvent.

In the methods described herein for the preparation of an oxine-containing cell radiolabelling agent, the agent is prepared by adding a solution containing a radioactive metal to an oxine formulation as described herein. In some embodiments the solution is an aqueous solution. In some embodiments the solution is an acidic solution. In some embodiments, the acidic solution comprises oxalic acid, hydrochloric acid, citric acid, tartaric acid, malonic acid, succinic acid or a combination thereof. In some embodiments the acidic solution comprises oxalic acid. In some embodiments the acidic solution consists of oxalic acid. A surprising advantage of using the formulations, methods and kits described herein is that a radioactive metal provided in an oxalic acid solution (for example, a commercial solution of ⁸⁹Zr-oxalate) does not need to be reformulated (e.g. in a chloride solution, a citrate solution, a tartrate solution, a malonate solution or a succinate solution) before the radioactive metal is added to the oxine formulation.

An oxine-containing cell radiolabelling agent, for example ⁸⁹Zr-oxine, ⁶⁸Ga-oxine, ⁶⁴Cu-oxine, ¹¹¹In-oxine, ⁶⁷Ga-oxine or ⁵²Mn-oxine, may be prepared by adding a solution containing the relevant radioactive metal to an oxine formulation as described herein.

In some embodiments of the methods described herein, an amount of radioactive metal providing between 0.1 MBq and 3000 MBq for every mg of oxine in the formulation is added to the oxine formulation. In some embodiments an amount of radioactive metal providing between 0.5 MBq and 800 MBq for every mg of oxine in the formulation is added. In some embodiments an amount of radioactive metal providing between 1.5 MBq and 700 MBq for every mg of oxine in the formulation is added. In some embodiments an amount of radioactive metal providing between 2 MBq and 600 MBq for every mg of oxine in the formulation is added.

In some embodiments of the methods described herein, an amount of radioactive metal providing between 100 MBq and 100000 MBq for every mmol of oxine in the formulation is added. In some embodiments, between 150 MBq and 90000 MBq of radioactive metal for every mmol of oxine in the formulation of the invention is added to the formulation. In some embodiments, between 200 MBq and 900 MBq of radioactive metal for every mmol of oxine in the formulation is added.

In some embodiments, after the addition of the radioactive metal containing solution to the formulation, the mixture is left for at least 15 minutes before use. In some embodiments, after the addition of the radioactive metal containing solution to the formulation the mixture is left for at least 10 minutes before use. In some embodiments, after the addition of the radioactive metal containing solution to the formulation the mixture is left for at least 5 minutes before use.

In some embodiments the step of adding a solution containing the radioactive metal to an oxine formulation as described herein takes place at room temperature. Room temperature can be defined as a temperature from 16° C. to 26° C. In some embodiments, the temperature is from 16 to 24° C. In some embodiments, the temperature is from 16 to 22° C. In some embodiments, the temperature is from 16 to 20° C. In some embodiments, the temperature is from 18 to 22° C. In some embodiments, the temperature is from 18 to 24° C. In some embodiments, the temperature is from 18 to 26° C. In some embodiments, the temperature is from 20 to 24° C.

In some embodiments, after the addition of the radioactive metal containing solution to the formulation, the mixture is filtered. In some embodiments, after the addition of the radioactive metal containing solution to the formulation, the mixture is not filtered.

Advantageously, in some embodiments the oxine formulation described herein can be freeze-dried, e.g. for storage or transport of the formulation or kit of the invention. The formulation can then be later reconstituted in a solvent (i.e. a solvent as described above) for use in the preparation of an oxine-containing radiolabelling agent, as described herein. Additionally, the formulation can advantageously be stored at room temperature.

Advantageously, when preparing oxine-containing cell radiolabelling agents by the method described herein, no subsequent neutralisation steps are required. Neutralisation steps are undesirable as they require meticulous precision by the end user, which is not conducive to the easy preparation of oxine-containing cell radiolabelling agent in a clinical setting.

In another aspect, provided herein is a quality control method for monitoring the formation of an oxine-containing cell radiolabelling agent. The method comprises the steps of: selecting an appropriate stationary phase, selecting an appropriate mobile phase, running a TLC (thin layer chromatography) assay, identifying peaks corresponding to unreacted radioactive metal, identifying peaks corresponding to formed oxine-containing cell radiolabelling agent.

In some embodiments a method as described herein further comprises a step of using TLC to calculate the yield of the oxine-containing cell radiolabelling agent formed. The yield is determined by measuring the areas under the curve (AUC) of the oxine-containing cell radiolabelling agent peak and the unreacted radioactive metal peak. The yield is then calculated by dividing the AUC of the oxine-containing cell radiolabelling agent by the sum of the AUC of both peaks and multiplying the result by 100.

In some embodiments of the method described herein the stationary phase is either instant thin-layer chromatography silica gel paper (ITLC-SG) or cellulose paper. In a preferred embodiment the stationary phase is cellulose paper. A benefit of using cellulose paper as the stationary phase is that a clear separation between the unreacted radioactive metal peak and the formed oxine-containing cell radiolabelling agent peak may be achieved. A preferred cellulose filter paper is Whatman® filter paper. A particularly preferred cellulose filter paper is Whatman® Grade 1 filter paper.

In some embodiments the pore size of the cellulose paper is between 0.1 µm and 100 µm. In some embodiments the pore size of the cellulose paper is between 1 µm and 30 µm. In some embodiments the pore size of the cellulose paper is about 11 µm. In some embodiments the length of the cellulose paper used in the method of the invention is between 1 and 10 centimetres. In a preferred embodiment the length of the cellulose paper used in the method is between 3 and 7 centimetres. In a more preferable embodiment the length of the cellulose paper used in the method is between 6 and 7 centimetres.

In some embodiments the mobile phase comprises ethyl acetate. In a preferred embodiment the mobile phase is 100% ethyl acetate.

In some embodiments the R_{f} value of the unreacted radioactive metal is 0. In some embodiments the R_{f} value of the formed oxine-containing cell radiolabelling agent is 0.8-1. In some embodiments the R_{f} value of the formed oxine-containing cell radiolabelling agent is 0.9-1.

The formulations, methods, kits and compositions described herein are suitable for use in a hospital radiopharmacy laboratory or clinical setting, for example in methods of cell radiolabelling and tracking, imaging methods, for example methods involving PET scanning or SPECT scanning, therapeutic methods (e.g. cell therapy and nanomedicine delivery) and diagnostic methods.

The formulations described herein may be used in all nuclear medicine departments performing conventional leukocyte scans (typically using scintigraphy or SPECT) and equipped with PET scanners, provided they have the shielding required for work with a positron and high energy gamma emitter.

In a typical clinical setup, a small amount of blood may be taken from a patient or suitable donor. Cells are isolated from this sample. Radiopharmacy staff reconstitute the formulation by adding water to a vial of a formulation as described herein, for example a freeze-dried formulation, and then adding 20 MBq of radioactive metal (e.g. ⁸⁹Zr) solution. Radiopharmacy staff can assess the formation of the relevant oxine-containing cell radiolabelling agent, e.g. according to the quality control method described herein, and may then use the formulation to radiolabel the cells. Non-cell-bound activity may then be removed from the cells, for example by centrifugation and/or washing. The radiolabelled cells may then be reinjected into the patient. After a suitable period of time, the patient may undergo a scan, for example a PET scan or SPECT scan. This may be performed, for example, to locate infections or inflammation.

Because the oxine formulation described herein is stable even after the addition of the radioactive metal, it may also be prepared (radiolabelled/reconstituted) in a central site and shipped as a radiopharmaceutical to other sites, for use without further processing.

More generally, a formulation, kit or composition as described herein will be useful in any clinical or preclinical study aimed at determining the fate of injected or implanted cells. For example, radiolabelled leukocytes can be used to understand their role in various diseases and to locate sites of infection or inflammation [30].

Furthermore, a formulation, kit or composition as described herein may be useful in advanced cell therapies, in particular using stem cells and/or therapeutic cells. An example of such use would be where a cancer patient undergoing CAR-T treatment is administered radiolabelled CAR-T in the first treatment round. A suitable imaging technique, for example a PET scan or SPECT scan would reveal the location and numbers of radiolabelled cells in the form of a 3D map and could potentially predict whether the patient would benefit from further treatment, or is at risk of side effects. Clinical decisions would be made based on imaging data, and the availability of a formulation as described herein will enable this procedure. Of immediate relevance, a clinical trial (T. Kalber) of mesenchymal stem cells has been approved and will take place in London in 2020-2021, Trial number: NCT03298763. This study includes a cohort of patients who will be administered cells labelled with ⁸⁹Zr-oxine. The formulations, kit or composition as described herein could potentially be used for this study.

In another aspect, provided herein is an oxine-containing cell radiolabelling agent prepared by the methods described herein.

In another aspect, provided herein is a method of labelling cells, liposomes or extracellular vesicles with the oxine-containing cell radiolabelling agent described herein comprising the step of incubating cells, liposomes or extracellular vesciles with the stabilised composition as described herein comprising: an oxine-containing cell radiolabelling agent; a surfactant; a base; a buffering agent; and a solvent.

In some embodiments, the composition is a stable composition of ⁸⁹Zr-oxine, ⁶⁸Ga-oxine, ⁶⁴Cu-oxine, ¹¹¹In-oxine, ⁶⁷Ga-oxine or ⁵²Mn-oxine. In some embodiments, the composition is a stable composition of ⁸⁹Zr-oxine, ⁶⁸Ga-oxine, or ⁶⁴Cu-oxine. In preferred embodiments, the composition is a stable composition of ⁸⁹Zr-oxine.

The composition is incubated with cells, liposomes or extracellular vesicles for a time sufficient to allow the labelling of the cells, liposomes or extracellular vesicles. In some embodiments of the invention the composition is incubated with the cells, liposomes or extracellular vesicles for between 10 and 60 minutes. In some embodiments of the invention the composition is incubated with the cells, liposomes or extracellular vesicles for between 10 and 30 minutes. In some embodiments of the invention the composition is incubated with the cells, liposomes or extracellular vesicles for between 15 and 25 minutes. In some embodiments of the invention the composition is incubated with the cells,r liposomes or extracellular vesicles for 20 minutes.

In some embodiments the method may further comprise the ‘one step’ method for preparing oxine-containing cell radiolabelling agents as described herein.

In some embodiments the method may further comprise the step of assessing the formation of the oxine-containing cell radiolabelling agent using the quality control method described herein.

In some embodiments, for example where the formulation is provided freeze-dried, the method may further comprise the step of reconstituting the formulation in a suitable solvent. In some preferred embodiments the solvent is water.

In some embodiments the cells are white blood cells (e.g. lymphocytes, NK cells, T cells, B cells, neutrophils, eosinophils, monocytes), red blood cells, platelets, liver cells, beta cells, bone marrow cells, therapeutic cells (e.g. CAR-T cells, gamma-delta T cells, tumour-infiltrating lymphocytes, dendritic cells, Treg cells, NK cells), stem cells (e.g. mesenchymal stem cells or haematopoietic stem cells) or tumour cells. In some embodiments the cells are cultured therapeutic cells. In some embodiments the cells are lymphocytes. In some embodiments the cells are CAR-T cells. In some embodiments the cells are gamma-delta T cells. In some embodiments the cells are donor cells. In some alternative embodiments the cells are stem cells. In some embodiments the cells are mesenchymal stem cells. In some embodiments the cells are haematopoietic stem cells. In some embodiments liposomes or extracellular vesicles are used instead of cells. In some embodiments the liposomes encapsulate a cargo e.g. a drug.

In some embodiments the method may further comprise the step of isolating cells from a blood sample.

In some embodiments the method may further comprise the step of extracting blood from a subject.

In some embodiments the method may further comprise the step of isolating cells from an expansion culture.

In another aspect, provided herein is a labelled cell prepared by the methods described herein.

In another aspect, provided herein is a labelled liposome prepared by the methods described herein.

In another aspect, provided herein is a labelled extracellular vesicle prepared by the methods described herein.

In another aspect, provided herein is a method of imaging comprising the steps of: incubating cells, liposomes or extracellular vesicles with the stable composition as described herein comprising: an oxine-containing cell radiolabelling agent, a surfactant, a base, a buffering agent and a solvent; administering cells or liposomes labelled with the oxine-containing cell radiolabelling agent as described herein to a subject; examining the subject using a suitable imaging technique, for example PET imaging or SPECT imaging.

In some embodiments, the composition is a stable composition of ⁸⁹Zr-oxine, ⁶⁸Ga-oxine, ⁶⁴Cu-oxine, ¹¹¹In-oxine, ⁶⁷Ga-oxine or ⁵²Mn-oxine. In some embodiments, the composition is a stable composition of ⁸⁹Zr-oxine, ⁶⁸Ga-oxine, or ⁶⁴Cu-oxine. In preferred embodiments, the composition is a stable composition of ⁸⁹Zr-oxine.

In some embodiments the cells, liposomes or extracellular vesicles labelled with the oxine-containing cell radiolabelling agent are administered to the subject by injection.

In some embodiments imaging is performed after 3 hrs. In some embodiments imaging is performed after 12 hrs. In some embodiments imaging is performed after 24 hrs. In some embodiments imaging is performed after 36 hrs. In some embodiments imaging is performed after 48 hrs. In some embodiments imaging is performed after 7 days. In some embodiments imaging is performed after 14 days. In some embodiments imaging is performed after 3 weeks. In some embodiments imaging is performed during or directly after the step of administering cells labelled with the oxine-containing cell radiolabelling agent described herein to a subject. In some embodiments imaging is performed continuously for a period of between 1 minute and 48 hrs.

In some embodiments the method further comprises the method of cell radiolabelling described herein.

In some embodiments the method further comprises the ‘one step’ method for preparing oxine-containing cell radiolabelling agents as described herein.

In another aspect, provided herein is a formulation, kit or composition as described herein for use in imaging.

In another aspect, provided herein is the use of a formulation, kit or composition as described herein for imaging.

In some embodiments the formulation, kit or composition is used in the imaging of cancer, sites of infection, sites of inflammation, sites of blood pooling or internal bleeding.

In another aspect, provided herein is a method of diagnosing infection or inflammation comprising: incubating cells with the stable composition as described herein comprising: an oxine-containing cell radiolabelling agent, a surfactant, a base, a buffering agent and a solvent; administering cells labelled with the oxine-containing cell radiolabelling agent as described herein to a subject; examining the subject using a suitable imaging technique, for example PET imaging or SPECT imaging; locating sites of infection or inflammation.

In some embodiments, the composition is a stable composition of ⁸⁹Zr-oxine, ⁶⁸Ga-oxine, ⁶⁴Cu-oxine or ¹¹¹In-oxine, ⁶⁷Ga-oxine or ⁵²Mn-oxine. In some embodiments, the composition is a stable composition of ⁸⁹Zr-oxine, ⁶⁸Ga-oxine, or ⁶⁴Cu-oxine. In preferred embodiments, the composition is a stable composition of ⁸⁹Zr-oxine.

In some embodiments the method further comprises the method of cell radiolabelling described herein.

In another aspect, provided herein is a formulation, kit or composition as described herein for use in diagnosing infection or inflammation.

In another aspect, provided herein is the use of a formulation, kit or composition as described herein for diagnosing infection or inflammation.

In some embodiments the formulation, kit or composition is used to diagnose infection or inflammation in bone, soft-tissue, an organ (e.g. kidney, lung or gall bladder) or a lymph node. In some embodiments the formulation, kit or composition is used to diagnose infection or inflammation in a vascular graft. In some embodiments the formulation, kit or composition is used to diagnose infection or inflammation in a prosthetic joint. In some embodiments the formulation, kit or composition is used to diagnose inflammatory bowel disease. In some embodiments the formulation, kit or composition is used to diagnose a urinary tract infection. In some embodiments the formulation, kit or composition is used to diagnose the cause of an unknown fever. In some embodiments the formulation, kit or composition is used to diagnose asthma. In some embodiments the formulation, kit or composition is used to diagnose a chronic obstructive pulmonary disease. In some embodiments the formulation, kit or composition is used to diagnose an autoimmune disease.

In another aspect, provided herein is a method of diagnosing blood pooling or internal bleeding comprising: incubating cells with the stable composition as described herein comprising: an oxine-containing cell radiolabelling agent, a surfactant, a base, a buffering agent and a solvent; administering cells labelled with the oxine-containing cell radiolabelling agent as described herein to a subject; examining the subject using a suitable imaging technique, for example PET imaging or SPECT imaging; locating sites of blood pooling or internal bleeding.

In some embodiments, the composition is a stable composition of ⁸⁹Zr-oxine, ⁶⁸Ga-oxine, ⁶⁴Cu-oxine or ¹¹¹In-oxine, ⁶⁷Ga-oxine or ⁵²Mn-oxine. In some embodiments, the composition is a stable composition of ⁸⁹Zr-oxine, ⁶⁸Ga-oxine, or ⁶⁴Cu-oxine. In preferred embodiments, the composition is a stable composition of ⁸⁹Zr-oxine. In other preferred embodiments, the composition is a stable composition of ⁶⁴Cu oxine.

In some embodiments the method further comprises the method of cell radiolabelling described herein.

In another aspect, provided herein is a formulation, kit or composition for use in diagnosing blood pooling or internal bleeding.

In another aspect, provided herein is the use of a formulation, kit or composition as described herein for diagnosing blood pooling or internal bleeding. In some embodiments the formulation, kit or composition is used to diagnose gastrointestinal bleeding. In some embodiments the formulation, kit or composition is used to diagnose lower gastrointestinal bleeding.

In another aspect, provided herein is a method of cell therapy comprising: incubating cells with the stable composition as described herein comprising: an oxine-containing cell radiolabelling agent, a surfactant, a base, a buffering agent and a solvent; administering cells labelled with the oxine-containing cell radiolabelling agent as described herein to a subject; examining the subject using a suitable imaging technique, for example PET imaging or SPECT imaging; locating the administered cells.

In some embodiments the cells are white blood cells (e.g. lymphocytes, NK cells, T cells, B cells, neutrophils, eosinophils, monocytes), red blood cells, platelets, liver cells, beta cells, bone marrow cells, T cells, therapeutic cells (e.g. CAR-T cells, gamma-delta T cells, tumour-infiltrating lymphocytes, dendritic cells, Treg cells, NK cells), stem cells (e.g. mesenchymal stem cells, haematopoietic stem cells, etc.) In some embodiments the cells are cultured therapeutic cells. In some embodiments the cells are lymphocytes. In some embodiments the cells are CAR-T cells. In some embodiments the cells are gamma-delta T cells. In some embodiments the cells are donor cells. In some alternative embodiments the cells are stem cells. In some embodiments the cells are mesenchymal stem cells. In some embodiments the cells are haematopoietic stem cells. In some embodiments the cell therapy treatment is T cell therapy treatment. In some preferred embodiments the cell therapy treatment is CAR-T treatment. In some alternative embodiments the cell therapy is gamma-delta T cell treatment. In some embodiments the cell therapy treatment is a cancer treatment. In some embodiments the cell therapy treatment is a stem cell treatment. In some embodiments the cell therapy treatment is mesenchymal stem cell therapy. In some embodiments the cell therapy treatment is haematopoietic stem cell transplantation.

In some embodiments, the composition is a stable composition of ⁸⁹Zr-oxine, ⁶⁸Ga-oxine, ⁶⁴Cu-oxine or ¹¹¹In-oxine, ⁶⁷Ga-oxine or ⁵²Mn-oxine. In some embodiments, the composition is a stable composition of ⁸⁹Zr-oxine, ⁶⁸Ga-oxine, or ⁶⁴Cu-oxine. In preferred embodiments, the composition is a stable composition of ⁸⁹Zr-oxine.

In another aspect, provided herein is a formulation, kit or composition for use in cell therapy treatments. In some embodiments the formulation, kit or composition can be used in stem cell treatments. In some embodiments the formulation, kit or composition can be used in mesenchymal stem cell therapy treatments. In some embodiments the formulation, kit or composition can be used in haematopoietic stem cell transplantation treatments. In some embodiments the formulation, kit or composition can be used in T cell therapy treatment. In some embodiments the formulation, kit or composition can be used in CAR-T cell treatments. In some alternative embodiments the formulation, kit or composition can be used in gamma-delta T cell therapy treatments. In some embodiments the formulation, kit or composition can be used in a cancer treatment.

In another aspect, provided herein is the use of a formulation, kit or composition as described herein for cell therapy. In some embodiments the formulation, kit or composition can be used in stem cell treatments. In some embodiments the formulation, kit or composition can be used in mesenchymal stem cell therapy treatments. In some embodiments the formulation, kit or composition can be used in haematopoietic stem cell transplantation treatments. In some embodiments the formulation, kit or composition can be used in T cell therapy treatment. In some embodiments the formulation, kit or composition can be used in CAR-T cell treatments. In some alternative embodiments the formulation, kit or composition can be used in gamma-delta T cell therapy treatments. In some embodiments the formulation, kit or composition can be used in a cancer treatment.

In another aspect, provided herein is a method of nanomedicine delivery comprising: incubating liposomes with the stable composition as described herein comprising: an oxine-containing cell radiolabelling agent, a surfactant, a base, a buffering agent and a solvent; administering liposomes labelled with the oxine-containing cell radiolabelling agent as described herein to a subject; examining the subject using a suitable imaging technique, for example PET imaging or SPECT imaging.

In some embodiments the liposomes encapsulate a cargo. In some embodiments the cargo is a drug. In some embodiments the drug is selected from a chemotherapy drug, an antibiotic or an anti-inflammatory drug. In some embodiments the drug is an anthracycline. In some embodiments the drug is doxorubicin. In some embodiments the drug is a bisphosphonate. In some embodiments the drug is alendronate. In some embodiments the drug is a glucocorticoid.

In some embodiments the method of nanomedicine delivery further comprises the step of identifying the location of the liposomes. In some embodiments the method of nanomedicine delivery further comprises the step of monitoring the clearance of liposomes. In some embodiments the method of nanomedicine delivery further comprises the step of assessing whether the liposomes reach a target.

In some embodiments, the composition is a stable composition of ⁸⁹Zr-oxine, ⁶⁸Ga-oxine, ⁶⁴Cu-oxine or ¹¹¹In-oxine, ⁶⁷Ga-oxine or ⁵²Mn-oxine. In some embodiments, the composition is a stable composition of ⁸⁹Zr-oxine, ⁶⁸Ga-oxine, or ⁶⁴Cu-oxine. In preferred embodiments, the composition is a stable composition of ⁸⁹Zr-oxine.

In some embodiments the method further comprises the method of cell radiolabelling described herein.

In another aspect, provided herein is a formulation, kit or composition as described herein for use in nanomedicine delivery.

In another aspect, provided herein is the use of a formulation, kit or composition as described herein for nanomedicine delivery.

In another aspect, provided herein is a method of nanomedicine delivery comprising: incubating extracellular vesicles with the stable composition as described herein comprising: an oxine-containing cell radiolabelling agent, a surfactant, a base, a buffering agent and a solvent; administering extracellular vesicles labelled with the oxine-containing cell radiolabelling agent as described herein to a subject; examining the subject using a suitable imaging technique, for example PET imaging or SPECT imaging.

In some embodiments the method of nanomedicine delivery further comprises the step of identifying the location of the extracellular vesicles. In some embodiments the method of nanomedicine delivery further comprises the step of monitoring the clearance of extracellular vesicles. In some embodiments the method of nanomedicine delivery further comprises the step of assessing whether the extracellular vesicles reach a target.

In some embodiments, the composition is a stable composition of ⁸⁹Zr-oxine, ⁶⁸Ga-oxine, ⁶⁴Cu-oxine or ¹¹¹In-oxine, ⁶⁷Ga-oxine or ⁵²Mn-oxine. In some embodiments, the composition is a stable composition of ⁸⁹Zr-oxine, ⁶⁸Ga-oxine, or ⁶⁴Cu-oxine. In preferred embodiments, the composition is a stable composition of ⁸⁹Zr-oxine.

In some embodiments the method further comprises the method of cell radiolabelling described herein.

In another aspect, provided herein is a method of diagnosing cancer comprising: incubating cells, liposomes or extracellular vesicles with the stable composition as described herein comprising: an oxine-containing cell radiolabelling agent, a surfactant, a base, a buffering agent and a solvent; administering cells, liposomes or extracellular vesicles labelled with the oxine-containing cell radiolabelling agent as described herein to a subject; examining the subject using a suitable imaging technique, for example PET imaging or SPECT imaging; locating sites of cancer.

In some embodiments, the composition is a stable composition of ⁸⁹Zr-oxine, ⁶⁸Ga-oxine, ⁶⁴Cu-oxine or ¹¹¹In-oxine, ⁶⁷Ga-oxine or ⁵²Mn-oxine. In some embodiments, the composition is a stable composition of ⁸⁹Zr-oxine, ⁶⁸Ga-oxine, or ⁶⁴Cu-oxine. In preferred embodiments, the composition is a stable composition of ⁸⁹Zr-oxine.

In some embodiments the method further comprises the method of cell radiolabelling described herein.

In another aspect, provided herein is a formulation, kit or composition for use in diagnosing cancer.

In another aspect, provided herein is the use of a formulation, kit or composition as described herein for diagnosing cancer.

In some embodiments the formulation, kit or composition is used to diagnose leukaemia, lymphoma, lung cancer, endocrine cancer, breast cancer, cervical cancer, ovarian cancer or head and neck cancer. In some preferred embodiments the the formulation, kit or composition is used to diagnose breast cancer.

In another aspect provided herein is an imaging method comprising the steps of: incubating cells. liposomes or extracellular vesicles with the stable composition as described herein comprising: an oxine-containing cell radiolabelling agent, a surfactant, a base, a buffering agent and a solvent; and examining the cells, liposomes or extracellular vesicles using a suitable imaging technique, for example PET imaging or SPECT imaging. Preferably, the imaging technique is PET imaging.

In some embodiments the imaging method is performed in vitro. In other embodiments, the imaging step is performed in vivo i.e. after radio-labelled cells have been administered to the patient.

In some embodiments, the composition is a stable composition of ⁸⁹Zr-oxine, ⁶⁸Ga-oxine, ⁶⁴Cu-oxine, ¹¹¹In-oxine, ⁶⁷Ga-oxine or ⁵²Mn-oxine. In some embodiments, the composition is a stable composition of ⁸⁹Zr-oxine, ⁶⁸Ga-oxine, or ⁶⁴Cu-oxine. In preferred embodiments, the composition is a stable composition of ⁸⁹Zr-oxine.

In some embodiments the method further comprises (i.e. is preceded by) the ‘one step’ method for preparing oxine-containing cell radiolabelling agents described herein.

As would be understood by the a person skilled in the art, although embodiments of the invention are described herein wherein one of the group of cells, liposomes or extracellular vesicles are labelled and/or used, it may equally be appropriate and possible to label and/or use a different member of the group of cells, liposomes or extracellular vesicles in those embodiments.

A benefit of the methods, kit and formulation of the invention disclosed herein is that the procedure for the operator in charge of radiolabelling cells is vastly simplified. This leads to shortened procedure time, reduced risk of error and reduced exposure of the operator to ionizing radiation. Another benefit of the methods, kit and formulation of the invention disclosed herein is that the oxine-containing cell radiolabelling agent can be readily prepared, without requiring specialised equipment. This allows procedures to be performed even in less well-equipped centres.The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/- 10%.

### EXAMPLES

The examples below are illustrative of particular embodiments and should not be construed as limiting the invention in any way.

### Example 1 - Quality Control Method for the Formation of Radiolabelled Oxine Complexes

Materials required:
Whatman no.1 filter paper, cut in strips of 1 cm x 8 cm
Ethyl acetate [141-78-6]
An appropriate vessel for thin-layer chromatography (TLC) development (e.g. glass cylinder, chromatography tank, 50 mL conical tube) sealable with a suitable lid.

In the selected vessel for TLC development, ethyl acetate was added so that the height of the liquid in the container remains below 1 cm. ⁸⁹Zr-oxine solution (1-2 µL) was spotted 1 cm away from the bottom edge of the Whatman no. 1 paper strip. A mark was made at 1 cm from the top edge of the paper strip. The paper strip was placed in the TLC vessel and allowed to develop until the ethyl acetate front reached the top mark. The paper strip was removed from the vessel, allowed to dry in air, and read on a linear TLC reader equipped with an appropriate probe for positron detection. ⁸⁹Zr-oxine was visualised as a peak close (R_{f} = 0.9-1) to the solvent front on the paper strip. Unreacted ⁸⁹Zr was visualised as a peak at the origin of the TLC strip (R_{f} = 0).

The areas under the curve of the ⁸⁹Zr-oxine peak and the unreacted ⁸⁹Zr peak were calculated and the formation yield was calculated by dividing the AUC of the ⁸⁹Zr-oxine peak by the sum of the AUC of both peaks and multiplying the result by 100.

For cell radiolabelling purposes, a yield of 80% or above is acceptable.

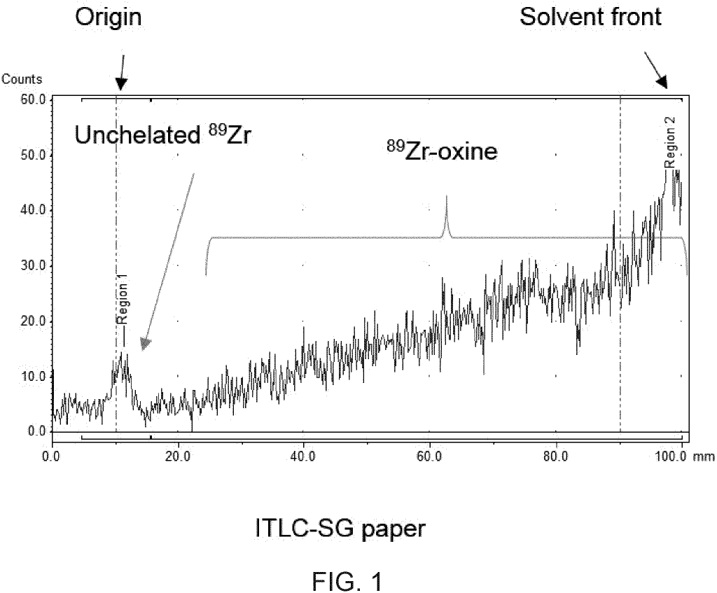

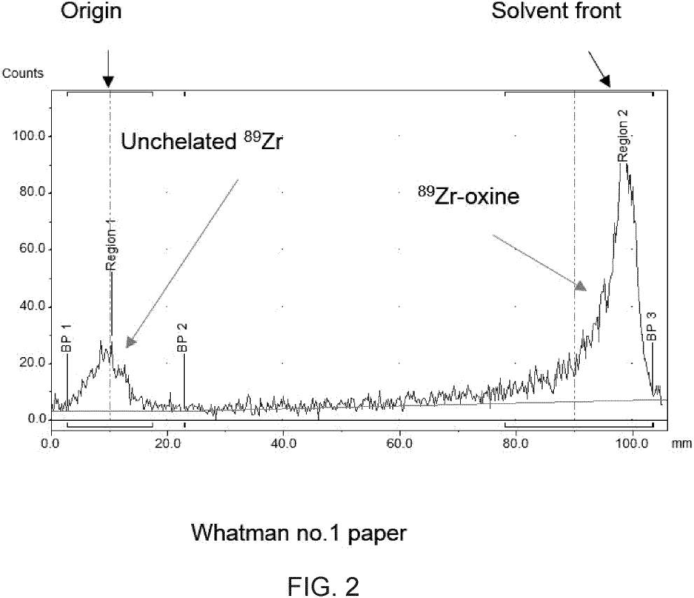

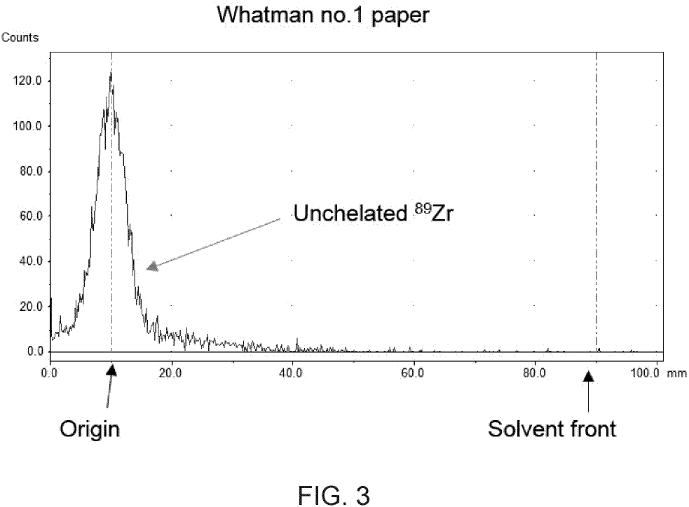

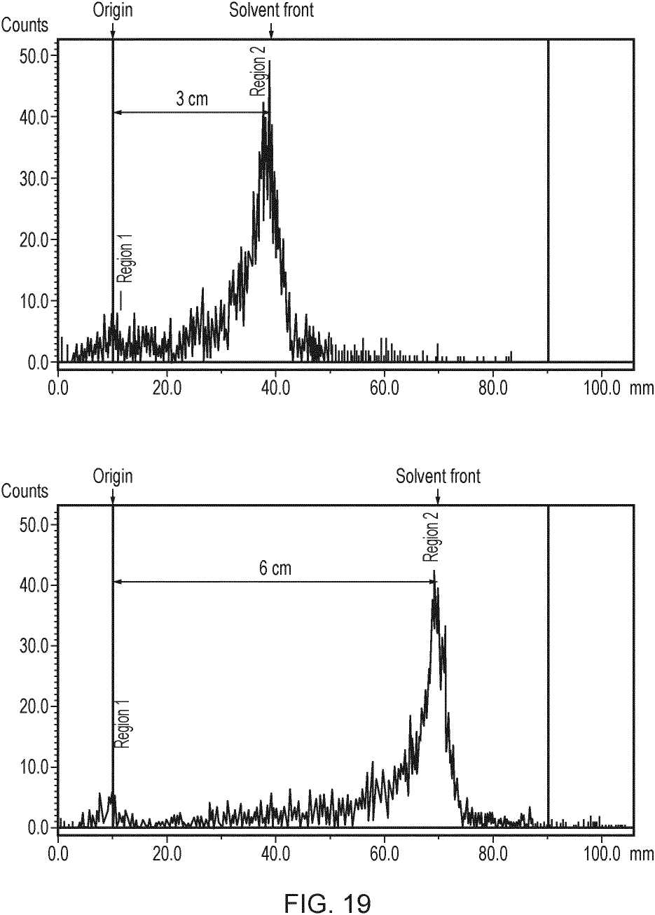

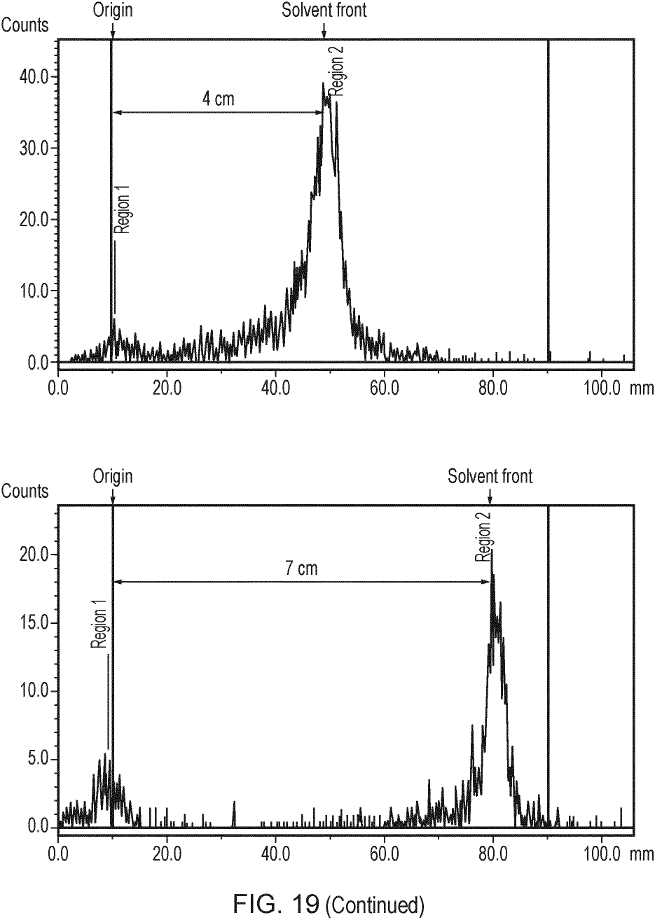

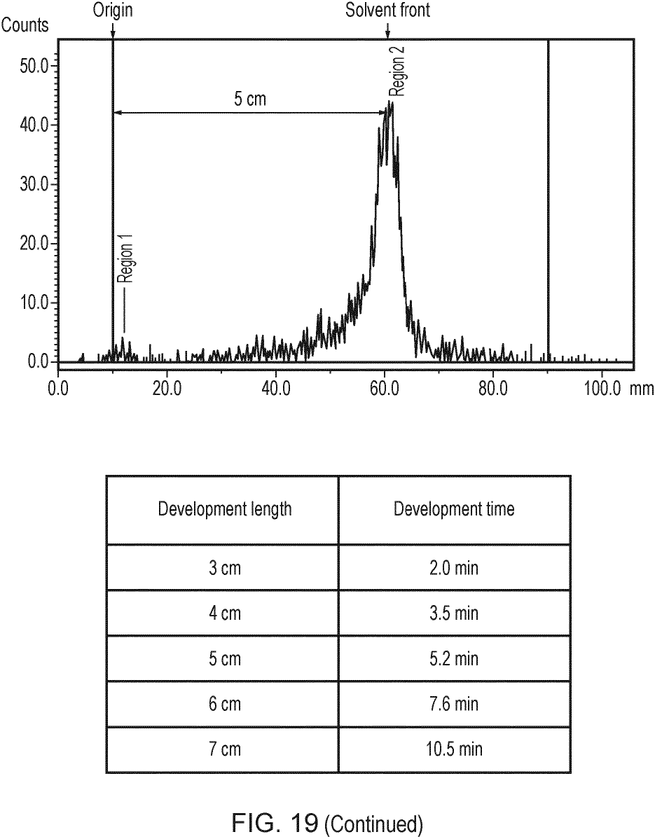

Radio-TLC of ⁸⁹Zr-oxine on silica-gel impregnated glass fibre (ITLC-SG) using ethyl acetate frequently showed marked streaking, possibly because the interaction of silanol groups with ⁸⁹Zr leads to the dissociation of the metastable ⁸⁹Zr-oxine complex during migration (FIG. 1). In contrast, TLC on Whatman no. 1 paper showed a clear separation between ⁸⁹Zr-oxine (R_{f} = 1, FIG. 2) and unchelated ⁸⁹Zr (R_{f} = 0, FIG. 3). A strip length of 6 cm was found to provide clear separation between the two species, with a migration time of less than 8 min (FIG. 19).

### Example 2- Testing of Activity Recovery and Stability of Radiolabelled Oxine Complexes

Activity recovery from vial: ⁸⁹Zr was added to 100 µL of the formulation. Aliquots of 10 µL were then retrieved immediately (reference sample) and after 15, 30, 60, 120 min, 24, 48, 72 and 168 h. The aliquots were gamma-counted on day 7 after addition of ⁸⁹Zr. The percentage of activity recovered from the vial was determined as the counts in each sample divided by the counts in the reference sample.

Stability of radiolabelled oxine complex: ⁸⁹Zr in 1 M oxalic acid was added to the formulation. A diluted formulation was obtained by further adding 900 µL H₂O. Samples were left at RT in the dark and stability was determined over 7 days by TLC as described in Example 1.

### Example 3 - Formulation Optimisation

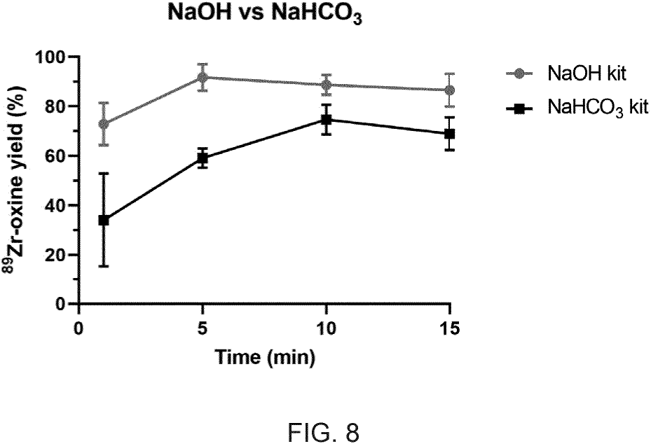

The formulation for preparing ⁸⁹Zr-oxine requires a base to neutralise the acidic solution of ⁸⁹Zr and a buffer to maintain the solution at pH 7-8. Alternative formulations were obtained by using NaHCO₃ (75 mmol) instead of NaOH, varying the amount of polysorbate 80 or replacing polysorbate 80 by EtOH (5% final concentration). It was found that 100 µL of HEPES-buffered formulation (containing 50 µg 8-hydroxyquinoline and 52.5 µmol NaOH) was capable of buffering (pH ≥ 7.0) a maximum of 18 µL of ⁸⁹Zr-oxalate solution. ⁸⁹Zr-oxine formulated in 1 M HEPES buffer (pH 7.9) in the absence of surfactant or organic solvent was found to rapidly adhere to glass vessels, with only 46% of the added activity recoverable from the vial 15 min after addition of ⁸⁹Zr. In the presence of 5% EtOH, 46% of the added ⁸⁹Zr activity was recoverable after 1 h, decreasing to less than 6% after 24 h (FIG. 9). Addition of 1 mg/mL polysorbate 80 to the formulation prevented adhesion to the glass vessel and resulted in 98.7% recovery of activity up to 1 week after addition of ⁸⁹Zr (FIG. 9). Reducing the amount of polysorbate 80 resulted in minor losses of product (FIG. 10). Using NaHCO₃ as an alternative to NaOH led to slower formation of ⁸⁹Zr-oxine and reduced yields (FIG. 8).

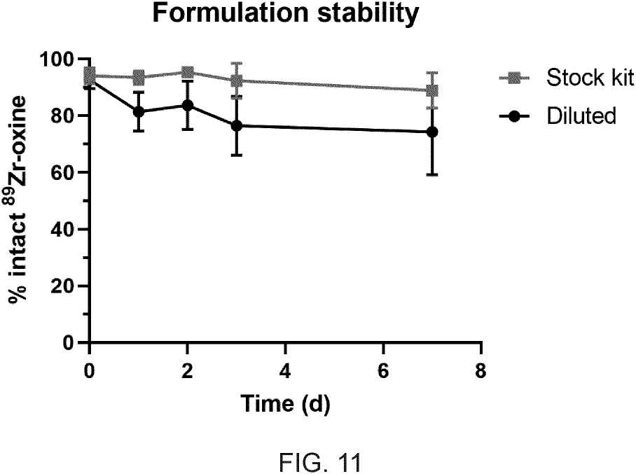

The optimised formulation (containing 1 M HEPES, NaOH and 1 mg/mL polysorbate 80, as described in Example 4) was stable for 7 days in concentrated format. Diluting the formulation to 1 mL with H₂O after addition of ⁸⁹Zr reduced the percentage of intact product from 92% to about 75% within 24-48 h (FIG. 11).

### Example 4a - Optimised Formulation Preparation

8-hydroxyquinoline (oxine, 50 mg, 3.44 mmol) was dissolved in 70 mL H₂O by heating at 75° C. for 10 min. After cooling to room temperature (RT), 23.83 g HEPES (100 mmol) was added, followed by 10 mL of a 10 mg/mL aqueous solution of polysorbate 80. The pH was adjusted to 8 by adding 10 M NaOH (52.5 mmol). The volume was adjusted to 100 mL with H₂O and the resulting solution filtered through a 0.2 µm membrane. This solution can be dispensed in 100 µL aliquots in glass vials and stored at RT in the dark or freeze-dried and reconstituted later with 0.1 mL H₂O.

### Example 4b - Alternative Optimised Formulation Preparation

8-hydroxyquinoline (oxine, 50 mg, 3.44 mmol) was added to 70 mL 0.1 M NaOH and agitated until complete dissolution. 23.83 g HEPES (100 mmol) was gradually added, followed by 10 mL of a 10 mg/mL aqueous solution of polysorbate 80. The pH was adjusted to 8 by adding 10 M NaOH (47 mmol). The volume was adjusted to 100 mL with H₂O and the resulting solution filtered through a 0.2 µm membrane. This solution can be dispensed in 500 µL aliquots in glass vials and stored at RT in the dark or freeze-dried. Each can be reconstituted later with 500 µL H₂O.

### Example 5 - Formation of a Radiolabelled Oxine Complex of ⁸⁹Zr

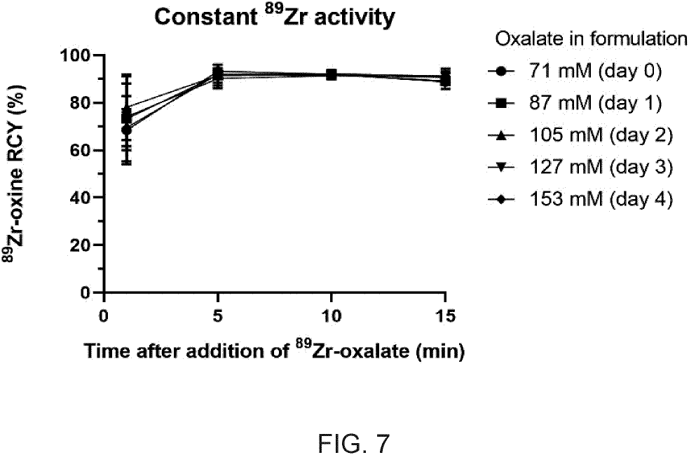

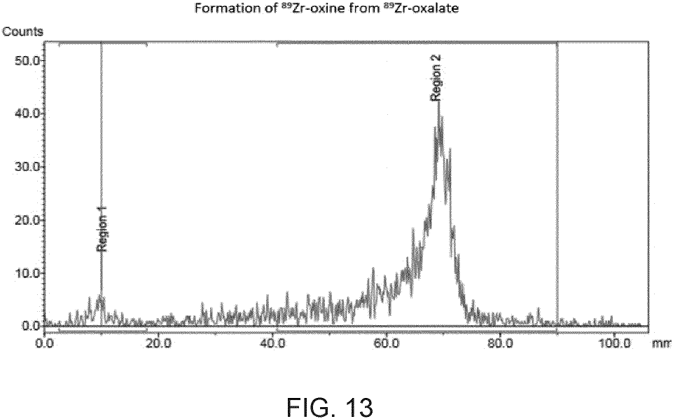

⁸⁹Zr in 1 M oxalic acid (1-18 µL, 0.5-25 MBq) was added to the formulation and left at RT for 5 min before use. Product formation was confirmed by TLC (FIGS. 2, 3, 13) as described in Example 1. When using ⁸⁹Zr-oxalate (1 M oxalic acid) as starting material, ⁸⁹Zr-oxine was typically formed in 90-92% radiochemical yield 5 min after addition of ⁸⁹Zr (FIG. 7). Increasing the volume of ⁸⁹Zr-oxalate up to a final oxalate concentration of 153 mM to account for ⁸⁹Zr decay did not affect the formation rate or final radiochemical yield of ⁸⁹Zr-oxine (FIG. 7).

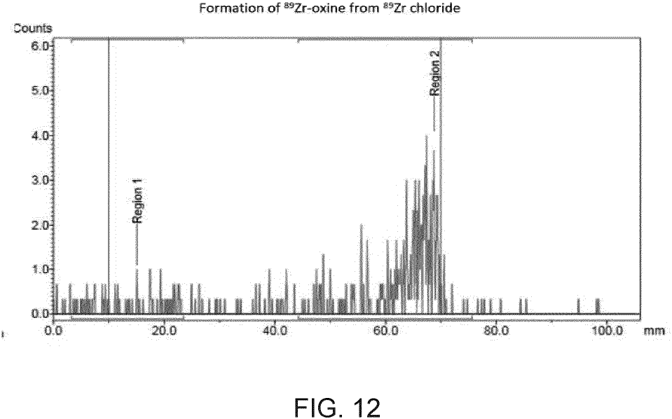

Alternatively, ⁸⁹Zr in 1 M hydrochloric acid can be added to the formulation (FIG. 12). ⁸⁹Zr in 1 M hydrochloric acid was obtained by loading commercial ⁸⁹Zr onto a Sep-Pak Light Plus QMA cartridge (Waters #WAT023525), washing the cartridge with 10 mL H₂O, and eluting with 500 µL of 1 M HCI [29].

### Example 6- Formation of a Radiolabelled Oxine Complexes of ⁶⁸Ga, ⁶⁴Cu and ¹¹¹In

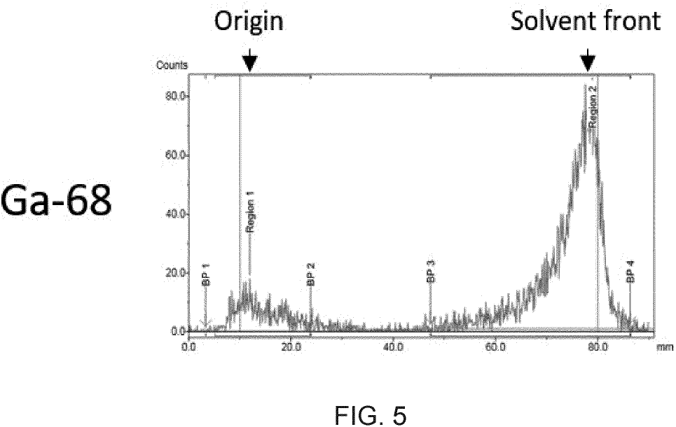

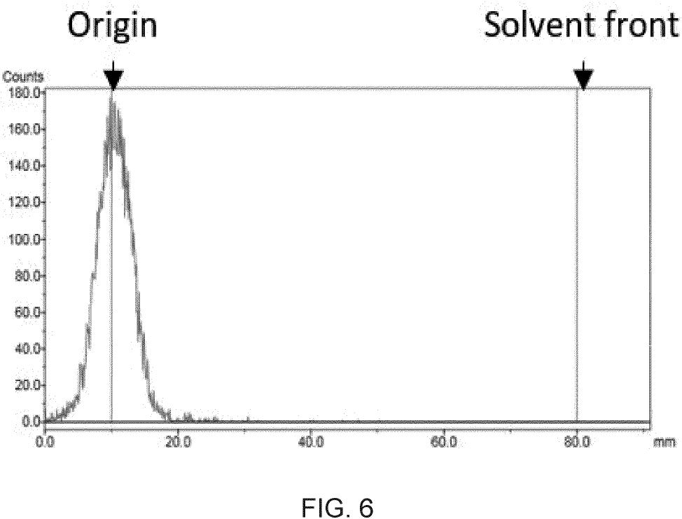

For ⁶⁸Ga-oxine: ⁶⁸Ga was eluted from a commercial generator using 0.1 M HCI, added to the formulation and left at RT for 5 min before use. Product formation was confirmed by TLC (FIGS. 5, 6, 20) using the method described in Example 1.

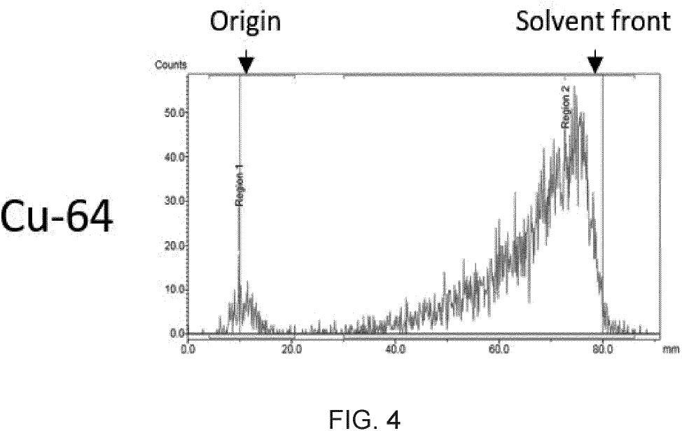

For ⁶⁴Cu-oxine: ⁶⁴Cu in 1-2 M HCI was added to the formulation and left at RT for 5 min before use. Product formation was confirmed by TLC (FIGS. 4, 6, 20) using the method described in Example 1.

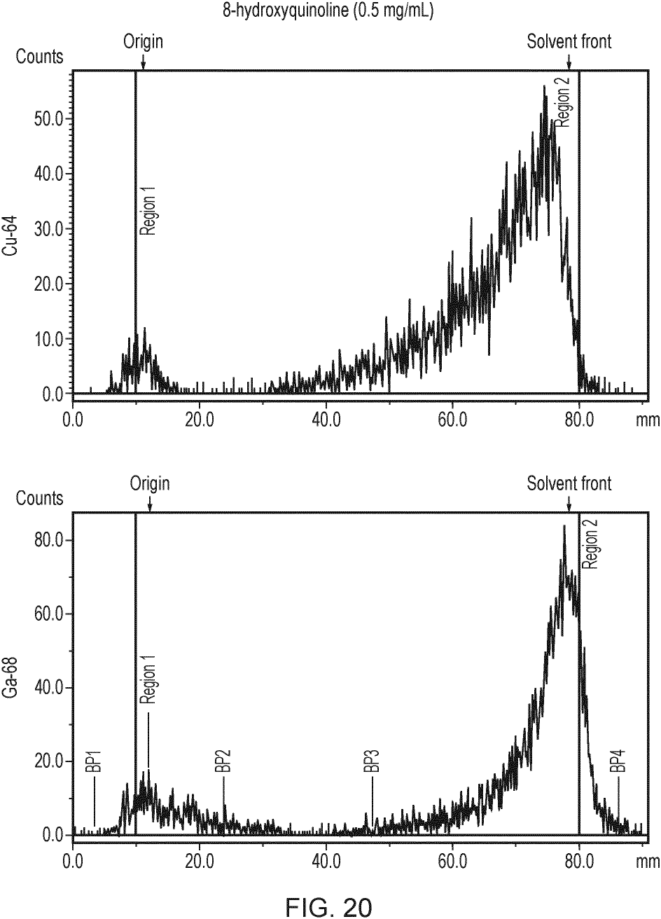

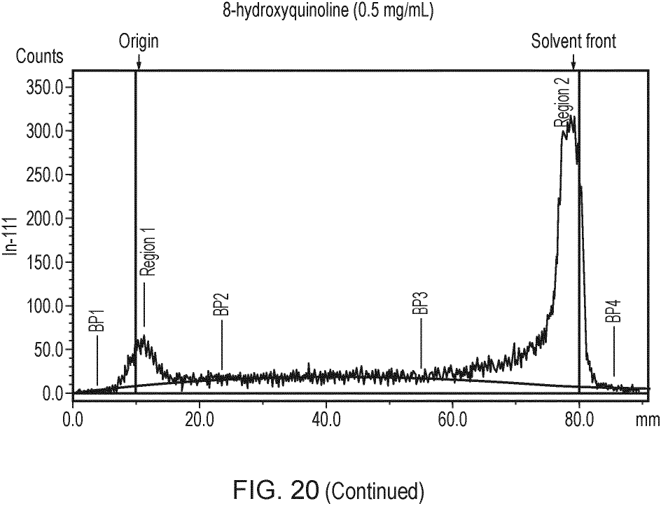

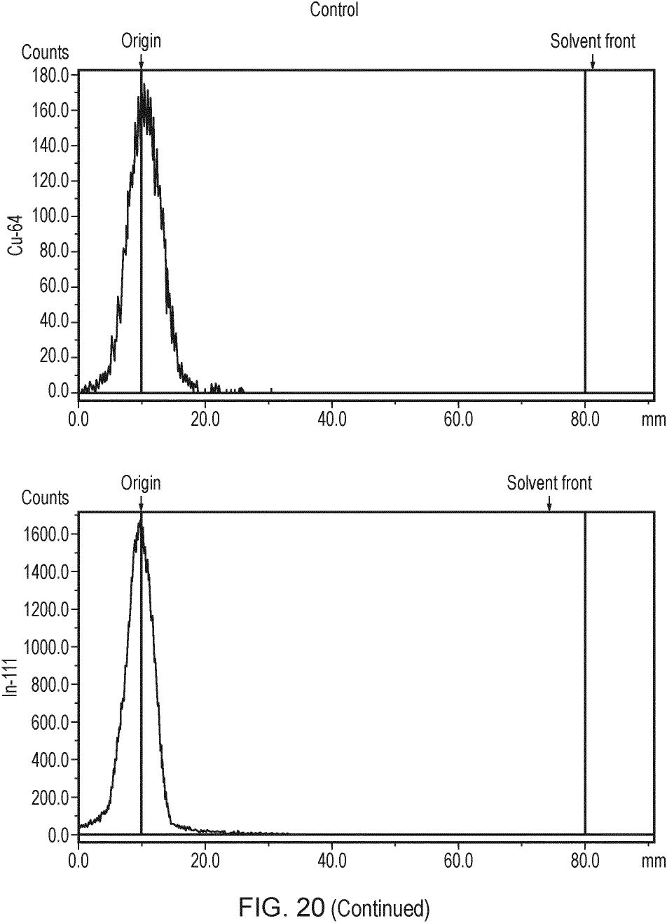

For ¹¹¹In-oxine, ¹¹¹In in 0.1 M HCI was added to the formulation and left at RT for 15 min before use. Product formation was confirmed by TLC (FIG. 20) using the method described in Example 1.

### Example 7- General Protocol for Radiolabelling Cells With Oxine Complexes

1. A radiolabelled oxine complex is prepared as described in Example 5 or Example 6.

2. Cells are isolated according to any suitable protocol and resuspended in 2-3 mL PBS or saline.

3. The radiolabelled oxine complex is added to the cell suspension and left to incubate at room temperature for 15-20 min with gentle swirling every 5 min. The total activity in the cell suspension is measured and recorded.

4. The cells are washed by addition of an appropriate medium followed by centrifugation at an appropriate speed for 10 min. The supernatant is decanted into a separate tube and the cell pellet is resuspended in an appropriate medium. The activities in the supernatant (Aₛₙ) and pellet (Aₚ) are measured and recorded. The labelling efficiency is calculated as 100 * (Aₚ)/(Aₚ + Aₛₙ).

5. The resuspended cells are subjected to standard Quality Control testing and further used in accordance with local protocols.

### Example 8- White Blood Cells Isolation and Radiolabelling With ⁸⁹Zr-Oxine and ¹¹¹In-Oxine

1. White blood cell isolation was performed in accordance with a clinical protocol used for radiolabelling WBC with ^{99m}Tc-exametazime. Briefly, peripheral blood (50-55 mL) was collected from healthy, male (n = 5) and female (n = 5) donors aged 22-32, in anticoagulant citrate dextrose solution A (ACD-A) blood collection tubes using 20G needles, on two separate occasions for each donor. Cell-free plasma (CFP) was obtained by centrifuging 10-15 mL blood at 2000 g for 10 min. For WBC isolation, 45 mL blood was mixed with 7 mL of HES200/0.5 (10% wt./vol. in sterile saline) and centrifuged at 8 g for 45 min at room temperature. Platelets were depleted by washing the WBC layer twice with Ca²⁺/Mg²⁺-free PBS (with 10 min centrifugation at 150 g). The remaining cell pellet (1.6-4.8×10⁸cells) was re-suspended in 3 mL PBS for radiolabelling.

2. A solution of ⁸⁹Zr-oxine containing 18-21 MBq ⁸⁹Zr was prepared as described in Example 5.

3. A solution of ¹¹¹In-oxine was prepared by adding ¹¹¹In in 0.1 M HCI (40-60 µL, 18-24 MBq) to 100 µL of a solution containing 50 µg 8-hydroxyquinoline, 100 µg polysorbate 80, 6 mg HEPES and 7.5 mg NaCl and adjusted to pH 7 with NaOH.

4. The ⁸⁹Zr-oxine or ¹¹¹In-oxine solutions were added to the cell suspension and left to incubate at room temperature for 15-20 min with gentle swirling every 5 min. As an additional control, an aliquot was incubated with PBS only.

5. The cells were washed by addition of 45 mL PBS followed by centrifugation at 200 g for 10 min. The supernatant was decanted into a separate tube and the cell pellet was resuspended in CFP or assay medium (RPMI-1640 supplemented with 1% human serum, 2 mM L-glutamine, 100 U/mL penicillin and 100 µg/mL streptomycin) for further experiments.

Each subject provided WBC on two separate occasions (at least 1 week apart), once for Zr-89 and once for In-111 labelling, enabling differences between groups to be evaluated by Student’s two-tailed, paired t-test. When additional factors were considered, analysis was performed using a repeated-measures Mixed Model (MM) in Prism v8.2 (GraphPad Software Inc.), with Tukey’s correction for multiple pairwise comparisons unless otherwise specified. Exact significance values are reported in each figure.

### Example 9 - Cell Radiolabelling Efficiency and Retention of Radiolabelling Agent

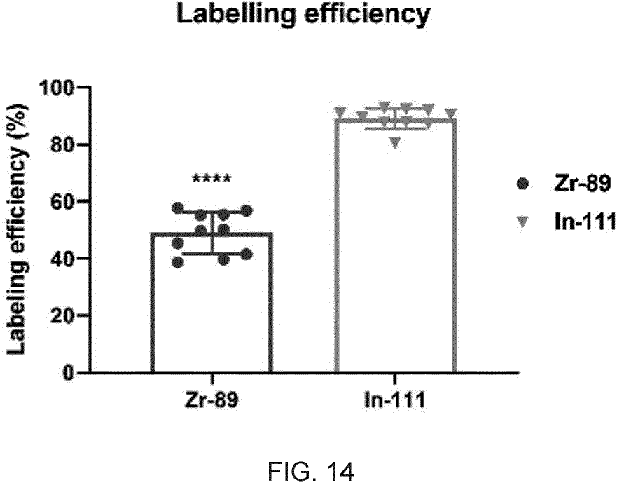

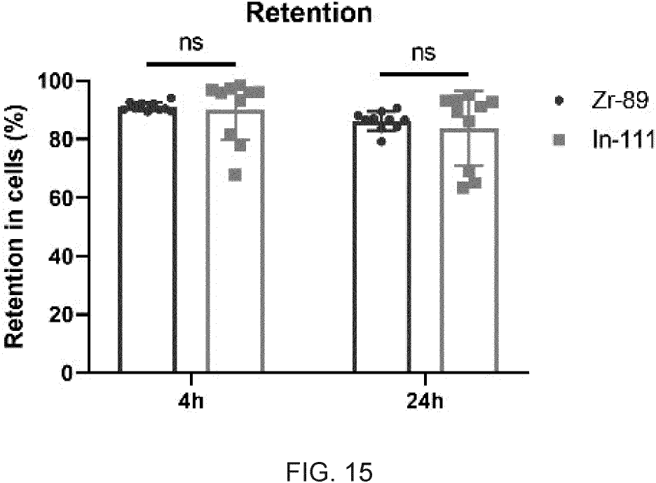

Human WBCs from 10 healthy donors were radiolabelled with ⁸⁹Zr-oxine and ¹¹¹In-oxine as described in Example 8 and an intra-individual comparison of labelling with ⁸⁹Zr-oxine and ¹¹¹In-oxine was performed. Labelling efficiency was determined as described in Example 7. For radiolabel retention studies, radiolabelled WBC were suspended in autologous CFP (cell free plasma), in triplicate in a 24-well plate and incubated at 37° C. Cells were collected after 4 h or 24 h, diluted with PBS, centrifuged at 200 g for 10 min, and supernatants and pellets were measured in a dose calibrator to determine retention using the same formula as for labelling efficiency. The labelling efficiency of WBCs with ⁸⁹Zr-oxine was 48.7±6.3%, vs 89.1±9.5 (p<0.0001, n=10) for ¹¹¹In-oxine (FIG. 14). Cellular retention of ⁸⁹Zr was 91.4±1.4% after 4 h incubation in autologous CFP and 86.6±2.9% after 24 h (FIG. 15). There were no significant differences between ⁸⁹Zr-oxine and ¹¹¹In-oxine for radiolabelling agent retention (p>0.05, n=10). The labelling of WBC with ⁸⁹Zr-oxine was reliable, consistently achieving 45-50% labelling efficiency with 160-480 million cells. Retention of ⁸⁹Zr-oxine in cells in the presence of plasma was high and comparable to that of ¹¹¹In-oxine, suggesting that radiotracer leakage will be low after intravenous administration and that the PET signal will accurately reflect the location of intact cells, as previously observed in preclinical studies [23].

### Example 10 - Determination of Viability of Radiolabelled Cells

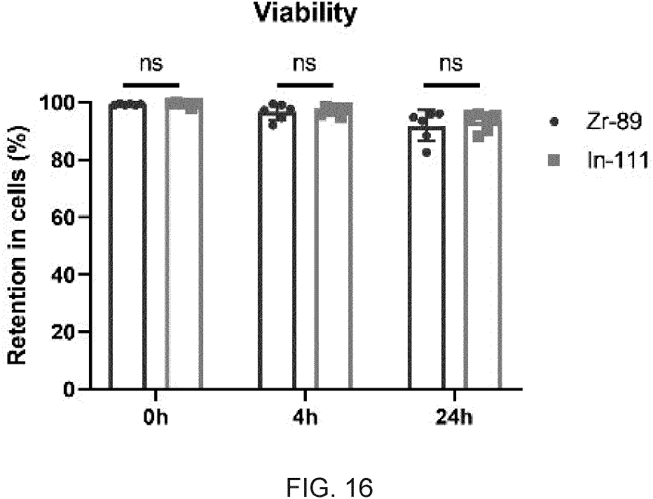

Viability was assessed by microscopy using the Trypan Blue dye exclusion method. Cell viability after radiolabelling with ⁸⁹Zr-oxine was 99.4±0.3% immediately after labelling, 97.0±2.3% after 4 h and 92.6±4.8% after 24 h (FIG. 16). There were no significant differences between ⁸⁹Zr-oxine and ¹¹¹In-oxine for viability (p>0.05, n=6-10). This further suggests that radiotracer leakage will be low after administration.

### Example 11 - Chemotaxis of Radiolabelled Cells

The functionality of WBCs radiolabelled as in Example 8 was tested using an in vitro chemotaxis assay, where the number of WBCs migrating in response to the neutrophil chemoattractant N-formyl-Met-Leu-Phe (fMLP) was measured.

After radiolabelling, red blood cells (RBCs) were removed from the cell pellets hypotonic lysis. Cells were resuspended in 4.5 mL cold H₂O for 30 s, after which isotonicity was restored by addition of 0.5 mL 10x PBS. Lysed RBC were removed by centrifugation and the remaining WBC were resuspended in assay medium at 3.5×10⁶ cells/mL. The bottom wells of a chemotaxis plate equipped with a polycarbonate membrane (3.2 mm diameter, 5 µm pore size) were filled with 30 µL of assay medium, with or without 10 nM fMLP. On the top wells, 20 µL of cell suspension were then plated in triplicate and incubated for 45 min at 37° C. Remaining cells in the top wells were removed, replaced by 40 µL of 5 mM EDTA in PBS to detach cells adhering to the membrane and the plate was incubated for 30 min at 4° C. The top wells were emptied, the plate was centrifuged at 150 g for 5 min to detach the cells from the membrane and the cells in the bottom wells were counted using a haemocytometer. The chemotaxis index (CI) was calculated by dividing the number of WBC in the wells containing fMLP by the number of WBC in the wells containing medium only.

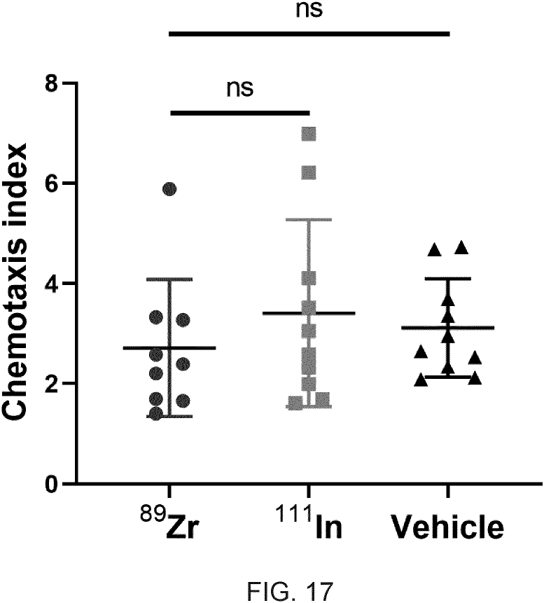

The chemotaxis index of ⁸⁹Zr-labelled WBCs was 2.7±1.4 (n=9), vs 3.4±1.9 (n=10) for ¹¹¹In-labelled WBCs and 3.0±1.0 (n=10) for non-labelled WBCs (FIG. 17). There were no significant differences between the groups. Importantly, the chemotaxis indexes were all >1, demonstrating an active migration towards fMLP. It is emphasized that leukocyte chemotaxis towards pathogens and inflammatory stimuli is the biological basis for WBC imaging. ⁸⁹Zr-labelled WBC retained their chemotactic properties in vitro, with no significant differences compared to ¹¹¹In-oxine and unlabelled control cells. This suggests that radiolabelling with ⁸⁹Zr-oxine will not affect in vivo properties of administered WBC at least within a few hours following radiolabelling and will provide clinically useful images.

### Example 12- Preferential Uptake of Radiolabels by Different Cell Types

To determine whether certain subtypes of WBC had preferential uptake of ⁸⁹Zr-oxine, radiolabelled WBC were stained with fluorescent monoclonal antibodies, automatically sorted and gamma-counted. ⁸⁹Zr-radiolabelled WBC were stained (15 min at 4° C.) with a combination of CD45-APC-Vio770 (#130-110-773), CD3-PE (REA613, #130-113-701), CD14-APC (#130-110-578), CD19-PE-Vio770 (REA675, #130-114-173) and CD16-FITC (REA423, #130-113-954) antibodies to sort samples into neutrophils, eosinophils, monocytes, T cells and B cells, or CD45-APC-Vio770, CD2354a-FITC (REA175, #130-117-800) and CD41a-PE (REA386, #130-121-429) to sort red blood cells and platelets. Compensation settings were adjusted using beads (anti-REA MACS® Comp, #130-104-693). Samples were analysed and sorted on a FACSMelody instrument (BD Biosciences) equipped with blue (488 nm), yellow-green (521 nm) and red (633 nm) lasers. For each cell type, a fixed number of events was collected, and the fractions were gamma-counted to determine the amount of activity per cell.

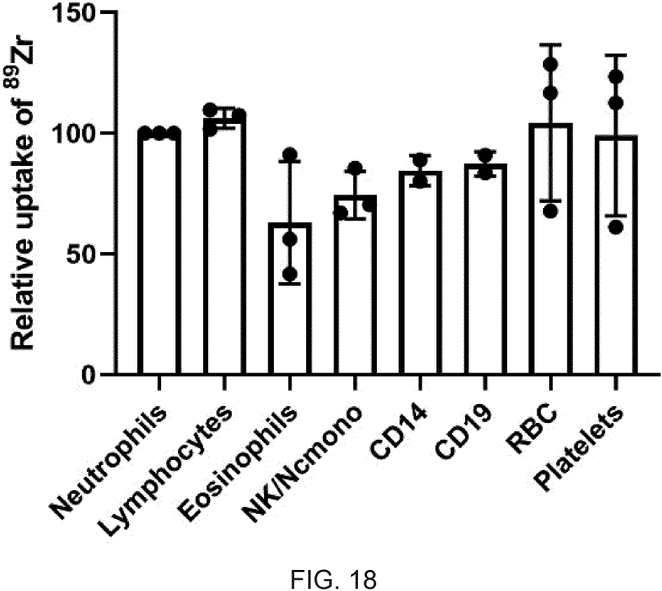

Results are expressed as relative activity per cell (FIG. 18). Taking neutrophils as reference (100%), the relative activity per cell of ⁸⁹Zr was 106±4% in lymphocytes, 63±25% in eosinophils, 74±10% in NK cells, 87±5% in B cells, 85±6% in monocytes, 99±33% in platelets and 104±32% in erythrocytes.

The results from the flow-assisted cell sorting indicate no preferential uptake by any specific leukocyte population. It has been shown previously that ^{99m}Tc-HMPAO accumulated preferentially in eosinophils [31], with the clinical implication that ^{99m}Tc-HMPAO WBC scans disproportionately represent the distribution of eosinophils. These results suggest that this phenomenon is not expected with ⁸⁹Zr-oxine. There is significant uptake in RBCs and platelets, therefore, better separation of these cells from WBC will improve signal specificity and target-to-background ratio.

### Example 13 - Imaging of Radiolabelled Cells in a Preclinical Model of Breast Cancer

Human breast cancer cells (HCC1954 cell line) were implanted in the mammary fat pad of female NSG mice (6-8 weeks old) on day 0. On days 12, 19 and 26, half of the mice were administered PEGylated liposomal alendronate (PLA, 1.3 mg/kg alendronate) intravenously. Human gammadelta T cells were isolated and expanded as previously described [23] and radiolabelled with ⁸⁹Zr-oxine as described in Example 8. On days 14 and 28, ⁸⁹Zr-labelled gammadelta T cells (10⁷ cells/mouse, 500 kBq per mouse) were injected intravenously (non-radiolabelled gammadelta T cells were injected on day 21). On days 16 and 30, the mice were imaged by PET/CT as previously described [23]. On days 63-64, the mice were culled and tumours were dissected. Tumours were fixed in formalin and embedded in paraffin for immunohistochemistry (IHC) analysis. Tumour sections were stained with haematoxylin and an antihuman CD3 antibody to detect human gammadelta T cells.

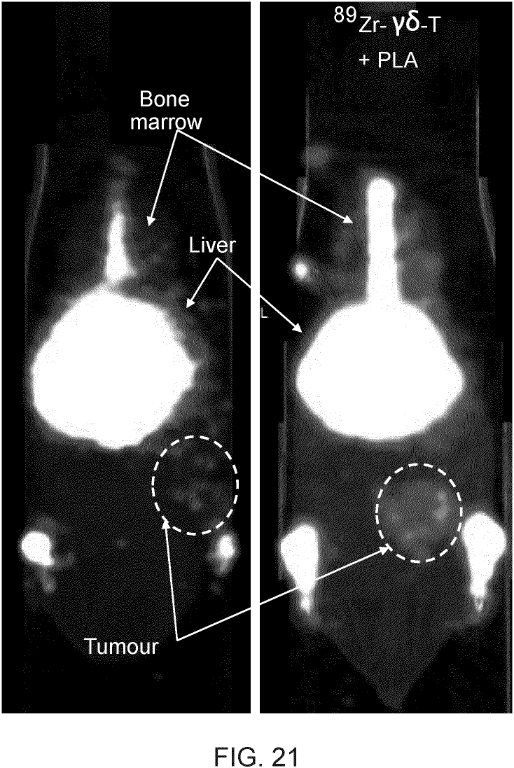

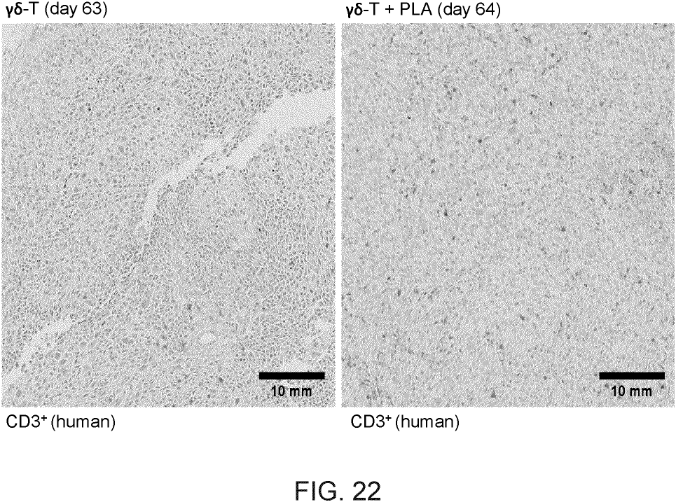

The PET/CT images show higher amounts of radioactivity in the tumour region on day 30 in mice treated with PLA compared to those without PLA, indicating increased migration of gammadelta T cells to the tumours after PLA treatment (FIG. 21). IHC images from samples taken on day 63-64 show CD3-positive cells (i.e. human gammadelta T cells) in tumours from mice treated both with and without PLA (FIG. 22). The images demonstrate that gammadelta T cells that migrate to the tumour can remain at the target site for at least 35 days after the last injection, proving durable engraftment.

### Example 14 - Radiolabelling of Extracellular Vesicles

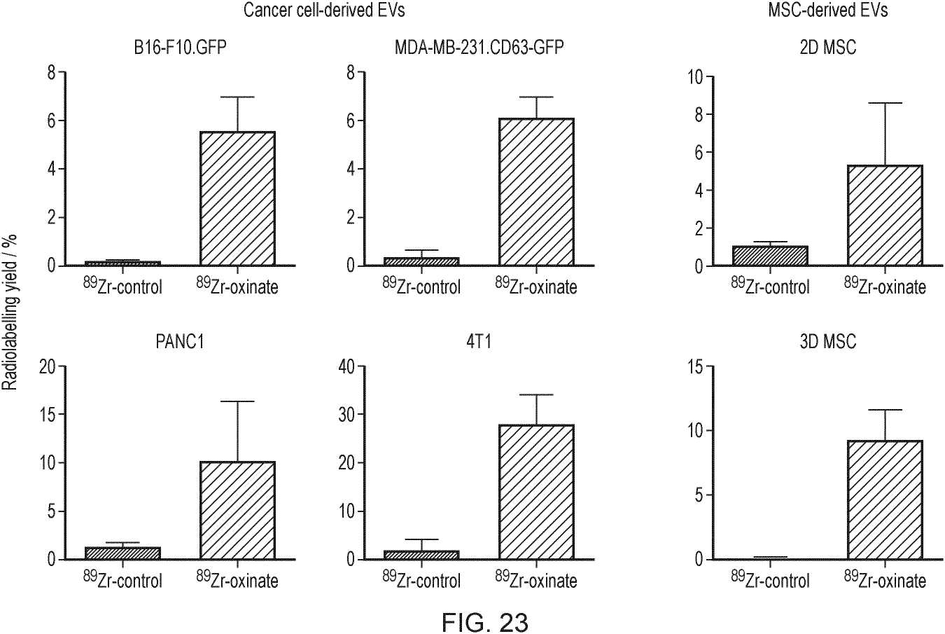

Extracellular vesicles are isolated by any suitable method (e.g. by density gradient centrifugation). Extracellular vesicles (e.g. 1×10¹⁰ to 1×10¹¹ vesicles) in 160 µL PBS were incubated with 20 µL ⁸⁹Zr-oxine or unchelated ⁸⁹Zr for 20 min at 37° C. with frequent shaking, followed by addition of 100 µL 1% deferoxamine in PBS to trap any unbound ⁸⁹Zr. Radiolabelled EVs were purified from unchelated radiotracer by size-exclusion chromatography (SEC) using cross-linked agarose resin (e.g. Sepharose CL-2B, GE Healthcare) in plastic columns. The reaction mixture was loaded onto the column followed by 200 µL PBS and eluted with 700-800 µL PBS. Radioactivity of the eluate and the column was measured using gamma counting to calculate labelling efficiency. The labelling efficiency of EVs obtained from B16-F10.GFP, MDA-MB-231.CD63-GFP, PANC1 and 4T1 cell lines and from 2D and 3D cultures of mesenchymal stem cells (MSC) B with ⁸⁹Zr-oxine ranged from 5.4 ± 3.2% for 2D-culture derived MSC-EVs to 27.6 ± 6.5% for 4T1 EVs (FIG. 23).

### Example 15 - Imaging of Radiolabelled Extracellular Vesicles in Mice

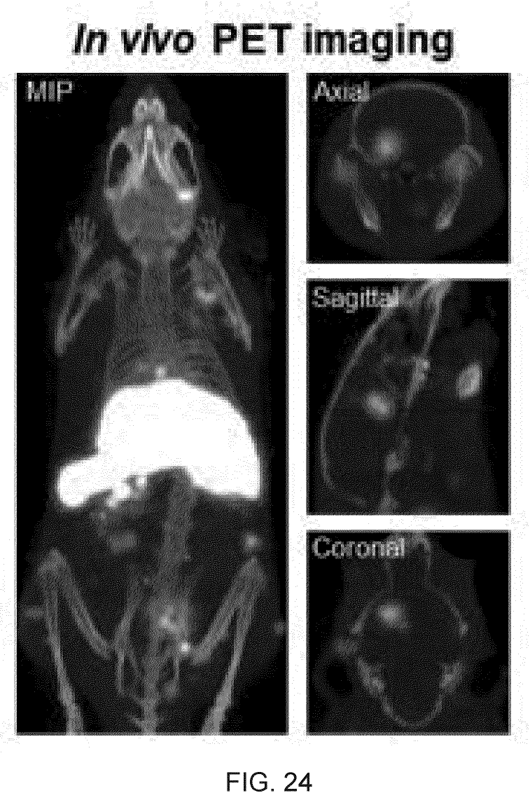

Immunocompetent C57BL/6j male mice (8-10 weeks old) were administered ⁸⁹Zr-labelled PANC1 EVs (0.2-1 MBq, approx. 1×10¹⁰ EVs in 100-140 µL PBS/mouse) intravenously. PET-CT imaging was performed on a nanoScan PET-CT preclinical imaging system (Mediso Medical Imaging System) 1 h after EV administration. The ⁸⁹Zr-labelled EVs were seen accumulating in the liver, spleen and in multiple lymph nodes (FIG. 24).

### REFERENCES

A number of publications are cited above in order to more fully describe and disclose the invention and the state of the art to which the invention pertains. Full citations for these references are provided below. The entirety of each of these references is incorporated herein.

C. J. Palestro, Radionuclide imaging of musculoskeletal infection: a review, J. Nucl. Med. 57 (2016) 1406-1412. doi.org/10.2967/jnumed.1 15.157297.

C.H. June, R.S. O’Connor, O.U. Kawalekar, S. Ghassemi, M.C. Milone, CAR T cell immunotherapy for human cancer, Science. 359 (2018) 1361-1365. doi:10.1126/science.aar6711.

A. Trounson, N.D. DeWitt, Pluripotent stem cells progressing to the clinic, Nat. Rev. Mol. Cell Biol. 17 (2016) 194-200. doi:10.1038/nrm.2016.10.

S. Chari, A. Nguyen, J. Saxe, Stem cells in the clinic, Cell Stem Cell. 22 (2018) 781-782. doi:10.1016/j.stem.2018.05.017.

G.P. Linette, E.A. Stadtmauer, M. V. Maus, A.P. Rapoport, B.L. Levine, L. Emery, L. Litzky, A. Bagg, B.M. Carreno, P.J. Cimino, G.K. Binder-Scholl, D.P. Smethurst, A.B. Gerry, N.J. Pumphrey, A.D. Bennett, J.E. Brewer, J. Dukes, J. Harper, H.K. Tayton-Martin, B.K. Jakobsen, N.J. Hassan, M. Kalos, C.H. June, Cardiovascular toxicity and titin cross-reactivity of affinity-enhanced T cells in myeloma and melanoma, Blood. 122 (2013) 863-871. doi:10.1182/blood-2013-03-490565.

M.L. James, S.S. Gambhir, A Molecular Imaging Primer: Modalities, Imaging Agents, and Applications, Physiol. Rev. 92 (2012) 897-965. doi:10.1152/physrev.00049.2010.

L.A. Forstrom, B.P. Mullan, J.C. Hung, V.J. Lowe, L.M. Thorson, 18F-FDG labelling of human leukocytes, Nucl. Med. Commun. 21 (2000) 685-690. https://doi.org/10.1097/00006231-200007000-00014.

K. Stojanov, E.F.J. de Vries, D. Hoekstra, A. van Waarde, R.A.J.O. Dierckx, I.S. Zuhorn, [18F] FDG labelling of neural stem cells for in vivo cell tracking with positron emission tomography: inhibition of tracer release by phloretin, Mol. Imaging. 11 (2012) 1-12. http://www.ncbi.nlm.nih.gov/pubmed/22418021.

E. Elhami, A.L. Goertzen, B. Xiang, J. Deng, C. Stillwell, S. Mzengeza, R.C. Arora, D. Freed, G. Tian, Viability and proliferation potential of adipose-derived stem cells following labelling with a positron-emitting radiotracer, Eur. J. Nucl. Med. Mol. Imaging. 38 (2011) 1323-1334. https://doi.org/10.1007/s00259-011-1753-9.

M.J. Welch, M.L. Thakur, R.E. Coleman, M. Patel, B.A. Siegel, M. Ter-Pogossian, Gallium-68 labelled red cells and platelets: new agents for positron tomography, J. Nucl. Med. 18 (1977) 558-62. http://www.ncbi.nlm.nih.gov/pubmed/404404.

C.M. Griessinger, R. Kehlbach, D. Bukala, S. Wiehr, R. Bantleon, F. Cay, A. Schmid, H. Braumuller, B. Fehrenbacher, M. Schaller, M. Eichner, J.L. Sutcliffe, W. Ehrlichmann, O. Eibl, G. Reischl, S.R. Cherry, M. Rocken, B.J. Pichler, M. Kneilling, In Vivo Tracking of Th1 Cells by PET Reveals Quantitative and Temporal Distribution and Specific Homing in Lymphatic Tissue, J. Nucl. Med. 55 (2014) 301-307. https://doi.org/10.2967/jnumed.113.126318.

N. Adonai, K.N. Nguyen, J. Walsh, M. Iyer, T. Toyokuni, M.E. Phelps, T. McCarthy, D.W. McCarthy, S.S. Gambhir, Ex vivo cell labeling with 64Cu-pyruvaldehyde-bis(N4-methylthiosemicarbazone) for imaging cell trafficking in mice with positron-emission tomography, Proc. Natl. Acad. Sci. 99 (2002) 3030-3035. https://doi.org/10.1073/pnas.052709599.

J.-J. Park, T.-S. Lee, J.-J. Son, K.-S. Chun, I.-H. Song, Y.-S. Park, K.-I. Kim, Y.-J. Lee, J.-H. Kang, Comparison of cell-labeling methods with 124 I-FIAU and 64 Cu-PTSM for cell tracking using chronic myelogenous leukaemia cells expressing HSV1-tk and firefly luciferase, Cancer Biother. Radiopharm. 27 (2012) 719-728. https://doi.org/10.1089/cbr.2012.1225.

Z.-B. Li, K. Chen, Z. Wu, H. Wang, G. Niu, X. Chen, 64Cu-labeled PEGylated polyethylenimine for cell trafficking and tumor imaging, Mol. Imaging Biol. 11 (2009) 415-423. https://doi.org/10.1007/s11307-009-0228-x.

K.K. Bhargava, R.K. Gupta, K.J. Nichols, C.J. Palestro, In vitro human leukocyte labeling with 64Cu: an intra-individual comparison with 111In-oxine and 18F-FDG, Nucl. Med. Biol. 36 (2009) 545-549. https://doi.org/10.1016/j.nucmedbio.2009.03.001.

L.K. Meszaros, P. Charoenphun, K. Chuamsaamarkkee, J.R. Ballinger, G.E.D. Mullen, T.J. Ferris, M.J. Went, P.J. Blower, 89Zr-Oxine Complex: a Long-Lived Radiolabel for Cell Tracking Using PET, World Mol. Imaging Congr. (2013). http://www.wmis.org/abstracts/2013/data/notes.htm?papers/LBAP024.htm?%27Zr-Oxine Complex%3A a Long-Lived Radiolabel for Cell Tracking Using PET%27 (accessed Jan. 14, 2020).

T.J. Ferris, P. Charoenphun, L.K. Meszaros, G.E.D. Mullen, P.J. Blower, M.J. Went, Synthesis and characterisation of zirconium complexes for cell tracking with Zr-89 by positron emission tomography, Dalton Trans. 43 (2014) 14851-14857. https://doi.org/10.1039/C4DT01928H.

P. Charoenphun, L.K. Meszaros, K. Chuamsaamarkkee, E. Sharif-Paghaleh, J.R. Ballinger, T.J. Ferris, M.J. Went, G.E.D. Mullen, P.J. Blower, [89Zr]oxinate4 for long-term in vivo cell tracking by positron emission tomography, Eur. J. Nucl. Med. Mol. Imaging. 42 (2015) 278-287. https://doi.org/10.1007/s00259-014-2945-x.

N. Sato, H. Wu, K.O. Asiedu, L.P. Szajek, G.L. Griffiths, P.L. Choyke, 89Zr-oxine complex PET cell imaging in monitoring cell-based therapies, Radiology. 275 (2015) 490-500. https://doi.org/10.1148/radiol.15142849.

K.O. Asiedu, S. Koyasu, L.P. Szajek, P.L. Choyke, N. Sato, Bone marrow cell trafficking analyzed by 89Zr-oxine positron emission tomography in a murine transplantation model, Clin. Cancer Res. (2016) 1-11. https://doi.org/10.1158/1078-0432.CCR-16-1561.

K.O. Asiedu, M. Ferdousi, P.T. Ton, S.S. Adler, P.L. Choyke, N. Sato, Bone marrow cell homing to sites of acute tibial fracture: 89Zr-oxine cell labeling with positron emission tomographic imaging in a mouse model, EJNMMI Res. 8 (2018) 109. https://doi.org/10.1186/s13550-018-0463-8.

M.R. Weist, R. Starr, B. Aguilar, J. Chea, J. Miles, E. Poku, E. Gerdts, X. Yang, S. Priceman, S. Forman, D. Colcher, C. Brown, J. Shively, Positron emission tomography of adoptively transferred chimeric antigen receptor T cells with Zirconium-89 oxine, J. Nucl. Med. 59 (2018) 1531-1537. https://doi.org/10.2967/jnumed.117.206714.

F. Man, L. Lim, A. Volpe, A. Gabizon, H. Shmeeda, B. Draper, A.C. Parente-Pereira, J. Maher, P.J. Blower, G.O. Fruhwirth, R. T.M. de Rosales, In vivo PET tracking of 89Zr-Labeled Vy9Vδ2 T Cells to mouse xenograft breast tumors activated with liposomal alendronate, Mol. Ther. 27 (2019) 219-229. https://doi.org/10.1016/j.ymthe.2018.10.006.

H.A. Watson, R.R.P. Durairaj, J. Ohme, M. Alatsatianos, H. Almutairi, R.N. Mohammed, M. Vigar, S.G. Reed, S.J. Paisey, C. Marshall, A. Gallimore, A. Ager, L-Selectin enhanced T cells improve the efficacy of cancer immunotherapy, Front. Immunol. 10 (2019). https://doi.org/10.3389/fimmu.2019.01321.

S. Edmonds, A. Volpe, H. Shmeeda, A.C. Parente-Pereira, R. Radia, J. Baguña-Torres, I. Szanda, G.W. Severin, L. Livieratos, P.J. Blower, J. Maher, G.O. Fruhwirth, A. Gabizon, R. T. M. de Rosales, Exploiting the metal-chelating properties of the drug cargo for in vivo positron emission tomography imaging of liposomal nanomedicines, ACS Nano. 10 (2016) 10294-10307. https://doi.org/10.1021 /acsnano.6b05935.

P.J. Gawne, F. Clarke, K. Turjeman, A.P. Cope, N.J. Long, Y. Barenholz, S.Y. Terry, R. T.M. de Rosales, PET imaging of liposomal glucocorticoids using 89Zr-oxine: theranostic applications in inflammatory arthritis, Theranostics. (2020). https://doi.org/10.7150/thno.40403.

M.A. Deri, B.M. Zeglis, L.C. Francesconi, J.S. Lewis, PET imaging with 89Zr: From radiochemistry to the clinic, Nucl. Med. Biol. 40 (2013) 3-14. https://doi.org/10.1016/j.nucmedbio.2012.08.004.

Larenkov, Bubenschikov, Makichyan, Zhukova, Krasnoperova, Kodina, Preparation of zirconium-89 solutions for radiopharmaceutical purposes: interrelation between formulation, radiochemical purity, stability and biodistribution, Molecules. 24 (2019) 1534. https://doi.org/10.3390/molecules24081534.

D.N. Pandya, N. Bhatt, H. Yuan, C.S. Day, B.M. Ehrmann, M. Wright, U. Bierbach, T.J. Wadas, Zirconium tetraazamacrocycle complexes display extraordinary stability and provide a new strategy for zirconium-89-based radiopharmaceutical development, Chem. Sci. 8 (2017) 2309-2314. https://doi.org/10.1039/C6SC04128K.

N. Farahi, C. Loutsios, N.Tregay, C.Summers, L.S. Lok, P.Ruparelia, C.K. Solanki, D.Gillett, E.R. Chilvers, and A.M. Peters, Radiolabelled leucocytes in human pulmonary disease, British medical bulletin. 127, no. 1 (2018) 69-82. https://doi.org/10.1093/bmb/ldy022

M.R.B. Puncher, P.J. Blower, Autoradiography and density gradient separation of technetium-99m-Exametazime (HMPAO) labelled leucocytes reveals selectivity for eosinophils, Eur. J. Nucl. Med. 21 (1994). https://doi.org/10.1007/BF00182350.

A. Khan, F. Man, F. Faruqu, J. Kim, F. Al-Salemee, A. Volpe, G.O. Fruhwirth, K.T. Al-Jamal, R. T.M. de Rosales, [⁸⁹Zr]Zr(oxinate)₄ Allows Direct Radiolabelling and PET Imaging of Small Extracellular Vesicles, ChemRxiv. Preprint. https://doi.org/10.26434/chemrxiv.12730463.v1

1. A method of preparing an oxine-containing cell radiolabelling agent, comprising the step of adding a solution containing a radioactive metal to a formulation comprising:
oxine;
a surfactant;
a base;
a buffering agent.

2. The method of claim 1 wherein the formulation comprises between 1 × 10⁻⁶ mmol and 0.07 mmol of oxine.

3. (canceled)

4. (canceled)

5. (canceled)

6. The method of claim 1, wherein the oxine-containing cell radiolabelling agent is ⁸⁹Zr-oxine.

7. (canceled)

8. The method of claim 1 wherein the base is a metal hydroxide.

9. (canceled)

10. The method of claim 1 wherein there is between 50 and 800 mmol of base per mmol of the oxine in the formulation of the invention.

11. (canceled)

12. (canceled)

13. (canceled)

14. (canceled)

15. The method of claim 1 wherein the surfactant is selected from polysorbate 80, dimethylsulfoxide, ethanol, or a combination thereof.

16. The method of claim 1 wherein there is between 0.01 and 20 mmol of surfactant per mmol of the oxine in the formulation.

17. (canceled)

18. (canceled)

19. (canceled)

20. (canceled)

21. The method of claim 1 wherein the buffering agent is selected from Tris (tris(hydroxymethyl)aminomethane), HEPES (4-(2-hydroxyethyl)-1-piperazineethanesulfonic acid), MOPS (3-(N-morpholino)propanesulfonic acid), PIPES (piperazine-N,N′-bis(2-ethanesulfonic acid)), ACES (2-(carbamoylmethylamino)ethanesulfonic acid), MOPSO (2-hydroxy-3-morpholin-4-ylpropane-1-sulfonic acid), Cholamine chloride hydrochloride (2-aminoethyl(trimethyl)azanium;chloride;hydrochloride), HEPPS (3-[4-(2-Hydroxyethyl)piperazin-1-yl]propane-1-sulfonic acid), glycinamide (2-Aminoacetamide), Glycylglycine (2-[(2-Aminoacetyl)amino]acetic acid), and HEPBS (N-(2-Hydroxyethyl)piperazine-N′-(4-butanesulfonic acid)).

22. The method of claim 1 wherein there is between 50 and 1500 mmol of buffer per mmol of the oxine in the formulation of the invention.

23. (canceled)

24. (canceled)

25. (canceled)

26. (canceled)

27. The method of claim 1 wherein the pH of the formulation is between 6.5 and 8.

28. (canceled)

29. (canceled)

30. (canceled)

31. The method of claim 1 wherein the solution containing a radioactive metal comprises oxalic acid, hydrochloric acid, citric acid, tartaric acid, malonic acid, succinic acid or a combination thereof.

32. (canceled)

33. (canceled)

34. A kit for the preparation of a stable composition of an oxine-containing cell radiolabelling agent, the kit comprising:
a) a formulation comprising:
oxine;
a surfactant;
a base;
a buffering agent;
b) optionally, instructions for use according to the method of claim 1.

35. (canceled)

36. (canceled)

37. A method of labelling cells, liposomes or extracellular vesicles with an oxine-containing cell radiolabelling agent, comprising the step of incubating cells, liposomes or extracellular vesicles with a composition comprising:
an oxine-containing cell radiolabelling agent;
a surfactant;
a base;
a buffering agent;
a solvent.

38. The method of claim 37 comprising the step of preparing said composition, by adding a solution containing a radioactive metal to a formulation comprising:
oxine;
a surfactant;
a base;
a buffering agent.

39. A method according to claim 37, further comprising the steps of:
administering cells,liposomes or extracellular vesicles labelled with the oxine-containing cell radiolabelling agent to a subject;
examining the subject using PET imaging.

40. (canceled)

41. The kit of claim 34, wherein the formulation comprises between 1 × 10⁻⁶ mmol and 0.07 mmol of oxine.

42. (canceled)

43. (canceled)

44. The the kit of claim 34, wherein the oxine-containing cell radiolabelling agent is ¹¹¹In-oxine, ⁸⁹Zr-oxine, ⁶⁸Ga-oxine, ⁶⁴Cu-oxine, ⁶⁷Ga-oxine or ⁵²Mn-oxine.

45. (canceled)

46. (canceled)

47. The the kit of claim 34, wherein the base is a metal hydroxide.

48. (canceled)

49. (canceled)

50. The kit of claim 34, wherein there is between 60 and 700 mmol of base per mmol of the oxine in the formulation.

51. (canceled)

52. (canceled)

53. (canceled)

54. The the kit of claim 34, wherein the surfactant is selected from polysorbate 80, dimethylsulfoxide, ethanol, or a combination thereof.

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)
